(19) United States
(12) Patent Application Publication
(10) Pub. No.: US 2016/0106516 A1
(43) Pub. Date: Apr. 21, 2016

(54) SYSTEMS FOR AUTOMATED BIOMECHANICAL COMPUTERIZED SURGERY

(71) Applicant: Sameh Mesallum, Boston, MA (US)

(72) Inventor: Sameh Mesallum, Boston, MA (US)

(21) Appl. No.: 14/724,306

(22) Filed: May 28, 2015

Related U.S. Application Data
(60) Provisional application No. 62/005,193, filed on May 30, 2014.

(51) Int. Cl. A61B 19/00

(52) U.S. Cl. CPC A61B 19/54; A61B 19/2203; A61B 19/50; A61B 19/201; A61B 2017/00017

(57) ABSTRACT

Provided are wearable devices that are compact, potable, and wearable or able to attach to a body part. The devices are configured to securely mate with body structures become one unit with the underlying body tissue to provide a relatively stable working surface. In one example, the sides of the device are constructed of a semi-rigid material with borders that conform to a body part. The semi-rigid wall can also conform (at least partially) to the working surface of a target body part and/or area to achieve a tight junction. The devices can also operate on non-uniform surfaces. Skin is not flat, thus the topography of said irregular surface can be scanned to provide a zero depth reference over the entire irregular surface. The zero depth reference enables management of surgical tools, print heads, etc. along the Z axis to provide precise operations regardless of the shape of the surface.

### RELATED APPLICATIONS

This application claims priority under 35 U.S.C. §119(e) to U.S. Provisional Patent Application Ser. No. 62/005,193 entitled “SYSTEMS FOR AUTOMATED BIOMECHANICAL COMPUTERIZED SURGERY,” filed May 30, 2014, which application is incorporated herein by reference in its entirety.

### BACKGROUND

Numerical control (NC) refers to the automation of machine tools that are operated by programmed commands which can be encoded on a storage device. Modern NC machines are programmed and executed independently from manual control (e.g., via hand wheels or levers), or mechanically automated via cams alone. Most NC machines are implemented as computer numerical control (CNC) machines.

In conventional CNC systems, end-to-end component design is highly automated using computer-aided design (CAD) and computer-aided manufacturing (CAM) programs. Often, the design programs are used to produce a computer file that is interpreted to extract the commands needed to operate a particular machine via a post processor. The file and/or commands can be loaded into a CNC machine for production. Since any particular component might require the use of a number of different tools—drills, saws, etc., modern machines often combine multiple tools into a single “cells.” In other installations, a number of different machines are used with an external controller and human or robotic operators that move the component from machine to machine. In either case, the series of steps needed to produce any part is highly automated and can be used to produce a component part, for example, of another machine that closely matches any design.

### SUMMARY

According to aspects and embodiments, a miniature form of CNC system is implemented to perform automated biomechanical surgical procedures. According to one embodiment, a miniature CNC machine can be attached to a body part or organ and execute programmatic instructions to perform a surgical procedure. In some examples, the miniature CNC machine is configured such that the z axis is defined by a surgical actuator (e.g., surgical device, needle, blade, staple, laser, etc.) and the surgical field for the procedure is defined by the working surface of the miniature CNC machine. According to one embodiment, the actuator moves in X, Y, and Z axes according to pre-programmed instructions that trigger movements of the surgical actuator. The instruction can include surgical operations to be performed at specific locations along the X, Y, and Z axes. In further embodiments, the surgical procedures executed by the miniature CNC machine can be controlled by an operator.

According to other embodiments, provided are wearable devices that are compact, portable, and wearable or able to attach to a body part. The devices are constructed and arranged to securely mate with body structures to become one unit with the underlying body tissue and provide a relatively stable working surface. In one embodiment, the sides of the device are constructed of a semi-rigid material with borders that conform to a body part on which the devices are to be attached. For example, semi-rigid sidewalls of the device are configured to conform (at least partially) to the working surface of a target body part and/or area to achieve a tight junction.

In further embodiments, the surgical devices or miniature CNC machines are configured to operate on non-uniform surfaces. For example, the working surface (e.g., area of skin) is not flat, so the topography of said irregular surface can be scanned to provide zero depth references over the entire irregular surface. The zero depth reference is used by the device to control operation of surgical tools, print heads, etc., along the Z axis to provide precise operations regardless of the shape (e.g., curvature) of the surface. In one example, the devices are configured to calculate surface elevations, detect and measure grooves, enabling the device to treat each measured point as if it is a horizontal flat surface where all points on said working surface are zero in the Z axis.

According to another embodiment, the devices are configured to recognize the progress of any execution of instructions. In one example, the device is configured to repetitively scan the working surface (e.g., target skin area), so that the device can restart a procedure from when the device was last used or an operation was last executed, even where the work surface has been changed. In some embodiments, the device is specially configured to print a large sized image piece by piece, where the device is moved along the body tissue as each portion of a print has been completed. The device can scan each completed element and determine what portions remain until the device has completed the entire print operation, covering the entire surface determined for the large image. In one example, the device is configured to find common pixels between two small images to continue adding more images (e.g., similar to creating a panoramic picture from multiple small overlapping pictures). In other examples, the device can print temporary reference points to continue a print job, or print reference points that will form a portion of a next image section.

According to another embodiment, the device is configured to correct for any movement or change in the orientation of the working surface in relation to an original position. For example, the device is configured to rescan the working surface frequently, and can further use a finished part of a printed image to re-orient the device and enable to the device to complete an original design file.

According to one aspect, a wearable surgical device is provided. The device comprises an actuator coupled to at least one tool, a plurality of motors for positioning the actuator in at least an x and y co-ordinate, a reference guide configured to establish a distance to a target surface, a driver operatively connected to the actuator for positioning the at least one tool in a z dimension, and programming instructions configured to position the actuator based on programmatic activation of the plurality of motors, position the tool based on programmatic activation of the driver, and execute a procedure on a target surface based on programmatic action of one or more or the plurality of motors, the driver, and the tool, wherein the programmatic action is determined responsive to the position defined by the reference guide.

According to various embodiments, the tool comprises a print head and the device further comprises an ink reservoir; the device executes programmatic instructions to print a first portion of an image on the target surface comprising a person's skin; the device further comprises a plurality of laser scanners; the device is configured to identify that the device has been attached to a new target surface and identify the first portion of the image responsive to signals received from the laser scanners; the device computes a second section of the image to print responsive to determining the new position relative to a former position or relative to the first portion of the image; the tool comprises a high frequency oscillating needle connected to an ink reservoir; the tool comprises a needle connected to an ink reservoir; the device is configured to generate an image at a subcutaneous position; the device is further configured to generate an image on a plurality of subcutaneous depths not visible to a human eye; the plurality of subcutaneous depths include at least a first image portion generated at a first layer depth and a second image portion generated at a second layer depth;

According to another aspect, a programmable surgical device is provided. The device comprises a surgical actuator coupled to at least one tool, a plurality of motors for positioning the surgical actuator in at least an x and y co-ordinate, an attachment member for fixing the surgical device in position over a bodily surface, a driver operatively connected to the surgical actuator for positioning the at least one tool in a z dimension, and programming instructions configured to position the surgical actuator based on programmatic activation of the plurality of motors, position the tool based on programmatic activation of the driver, and execute a procedure at a depth defined by the z dimension based on programmatic action of one or more or the plurality of motors, the driver, and the surgical tool.

According to various embodiments, the device further comprises a reference guide configured to establish a depth reference on a target surface; the reference guide comprises a skid plate; the reference guide comprises a guide wheel; the guide wheel is deployable from a surgical actuator and is responsive to contact with the target surface; the device further comprises a base portion for contacting a body surface or tissue surface; the attachment member is coupled to the base portion; the attachment component comprises at least one strap extensible about a body part; the base portion further comprises an opening, and wherein the surgical tool access the body surface or the tissue surface through the opening to execute the surgical procedure; the device further comprises a second surgical actuator coupled to at least a second tool; the device further comprises a respective plurality of motors for positioning the second surgical actuator in at least an x and y plane, and a second driver operatively connected to the second surgical actuator for positioning the at least the second surgical tool in a z plane; the device further comprises a plurality of tools housed in a storage portion of the device; the tools comprising at least one, of a print head, a high frequency oscillating needle, scalpel, suture, needle and thread, stapler; the programming instructions are further configured to position the surgical actuator to release the surgical tool in the storage portion of the device; the programming instructions are further configured to couple the surgical actuator to a different surgical tool; the programming instructions are further configured to move a scalpel through a volume of tissue to be removed.

According to another aspect provided are embodiments of computer implemented methods for executing each individual function and/or steps for controlling each individual element. In further embodiment, each combination of individual functions and/or steps for controlling each individual element are combined into selections of one, two, three, four, five, six, seven, eight, nine, ten, eleven, and twelve of the individual elements.

Still other aspects, embodiments and advantages of these exemplary aspects and embodiments, are discussed in detail below. Moreover, it is to be understood that both the foregoing information and the following detailed description are merely illustrative examples of various aspects and embodiments, and are intended to provide an overview or framework for understanding the nature and character of the claimed aspects and embodiments. Any embodiment disclosed herein may be combined with any other embodiment. References to “an embodiment,” “an example,” “some embodiments,” “some examples,” “an alternate embodiment,” “various embodiments,” “one embodiment,” “at least one embodiment,” “this and other embodiments” or the like are not necessarily mutually exclusive and are intended to indicate that a particular feature, structure, or characteristic described in connection with the embodiment may be included in at least one embodiment. The appearances of such terms herein are not necessarily all referring to the same embodiment.

### BRIEF DESCRIPTION OF THE DRAWINGS

Various aspects of at least one embodiment are discussed below with reference to the accompanying figures, which are not intended to be drawn to scale. The figures are included to provide an illustration and a further understanding of the various aspects and embodiments, and are incorporated in and constitute a part of this specification, but are not intended as a definition of the limits of any particular embodiment. The drawings, together with the remainder of the specification, serve to explain principles and operations of the described and claimed aspects and embodiments. In the figures, each identical or nearly identical component that is illustrated in various figures is represented by a like numeral. For purposes of clarity, not every component may be labeled in every figure. In the figures:

FIG. 1A is a partially transparent view of a programmable surgical device, according to one example;

FIG. 1B is a partially transparent view of a programmable surgical device with multiple operating axes, according to one example;

FIG. 2A is a partially transparent view of a programmable surgical device contoured to conform to a curved surface, according to one example;

FIG. 2B is a partially transparent view of a programmable surgical device, according to one example;

FIG. 3 is a partially transparent view of a programmable surgical device, according to one example;

FIG. 4A is a partially transparent view of tracking areas around surgical field, according to one example;

FIG. 4B is a partially transparent view of a programmable surgical device, according to one example;

FIG. 5A is a partially transparent view of a programmable surgical device, according to one example;

FIG. 5B is a partially transparent view of a programmable surgical device, according to one example;

FIG. 6 is a partially transparent view of a programmable surgical device, according to one example;

FIGS. 7A-B are partially transparent views of a programmable surgical device, according to some examples;

FIGS. 8A-B are partially transparent views of a programmable surgical device, according to some examples;

FIGS. 9A-C illustrate example axes of operation for examples of a programmable surgical device;

FIGS. 10A-B, 11-12 are partially transparent views of a programmable surgical device, according to one example;

FIGS. 13-17 show various examples of a programmable surgical device;

FIG. 18 illustrates an example printing, according to one example;

FIG. 19 is a programmable surgical device, according to one example;

FIG. 20 illustrates a subcutaneous image generates by a programmable surgical device;

FIGS. 21-25 show various examples of a programmable surgical device;

FIG. 26A-D are views of a programmable surgical device configured for image printing and/or tattooing of images on a skin surface;

FIGS. 27A-B show examples of a programmable surgical device having optional ink reservoirs;

FIG. 28 illustrates the operation of a guide wheel, according to one example;

FIGS. 29, 30A-B, and 31 show examples of a programmable surgical device; and

FIG. 32 is a block diagram of a special purpose computer system configured to perform processes and functions disclosed herein;

FIGS. 33A-B are example process flows for executing procedures on target body surfaces.

### DETAILED DESCRIPTION OF THE INVENTION

Stated broadly various aspects of the invention are directed to miniature CNC machines that are constructed and arranged for engagement with body parts and/or organs. According to some embodiments, the miniature CNC operates as programmable surgical platform or programmable surgical device that can be fixed to a body part or body surface. The programmable surgical device is configured to scan and identify a surgical topography, for example, based on scanning components disposed on the surgical device. In one embodiment, the surgical device can include optical scanners, ultra-sound scanners, infra-red scanners, as well as ultra-violet scanners, among other options and/or combinations. The surgical device can be attached to a body part and/or organ by anchoring elements. Once anchored, the scanning components can be configured to identify a surgical field and/or track any motion of the surgical device with respect to the body part, surgical field, and/or organ. In some examples, the surgical device is programmed to compensate for motion of the device with the respect to the identified surgical field.

According to one embodiment, the surgical device can include a plurality of surgical implements to execute a variety of surgical procedures. In some examples, the surgical device moves the surgical device along X and Y axes defined within the surgical field. The device can execute surgical operations along a Z axis, for example, deploying the surgical implement along the Z axis and into a body part and/or organ. In some embodiments, the surgical device is programmed to change tools such that surgical procedures can be executed according to multiple stages of surgery. For example, each stage can include execution of one or more procedures at one or more co-ordinate spaces (e.g., various x, y, and z co-ordinates). Each stage can be followed by a tool change procedure wherein a surgical device is exchanged for another. A next or subsequent stage can then continue a surgical program by executing a next stage.

Examples of the methods, devices, and systems discussed herein are not limited in application to the details of construction and the arrangement of components set forth in the following description or illustrated in the accompanying drawings. The methods and systems are capable of implementation in other embodiments and of being practiced or of being carried out in various ways. Examples of specific implementations are provided herein for illustrative purposes only and are not intended to be limiting. In particular, acts, components, elements and features discussed in connection with any one or more examples are not intended to be excluded from a similar role in any other examples.

Also, the phraseology and terminology used herein is for the purpose of description and should not be regarded as limiting. Any references to examples, embodiments, components, elements or acts of the systems and methods herein referred to in the singular may also embrace embodiments including a plurality, and any references in plural to any embodiment, component, element or act herein may also embrace embodiments including only a singularity. References in the singular or plural form are not intended to limit the presently disclosed systems or methods, their components, acts, or elements. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms.

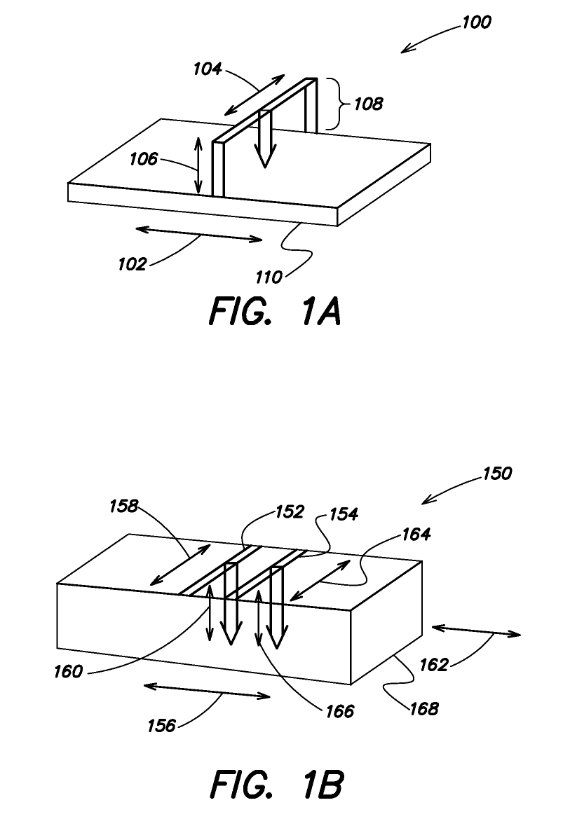

FIG. 1A is a partially transparent view 100 of a programmable surgical device. The view 100 illustrates the X axis 102, Y axis 104, and Z axis 106 through which a surgical actuator can be positioned with respect to a body part surface or organ surface (e.g., 110). According to some embodiments, the programmable surgical device is constructed and arranged with a flat configuration for a respective surgical field. Other embodiments can be configured with surgical fields that conform to contours of body parts and/or organs.

FIG. 1B is a partially transparent view 150 of a programmable surgical device having two surgical actuators 152 and 154 and respective X axis, Y axis, and Z axis (e.g., 156, 158, 160 and 162, 164, and 166, respectively) associated with a body part and/or organ surface (e.g., 168). According to some embodiments, the programmable surgical device can be constructed with a flat configuration for positioning on flat surgical fields of target body parts and/or organs. In various embodiments, the system uses one or two z-axes to perform tasks (e.g., surgical tasks).

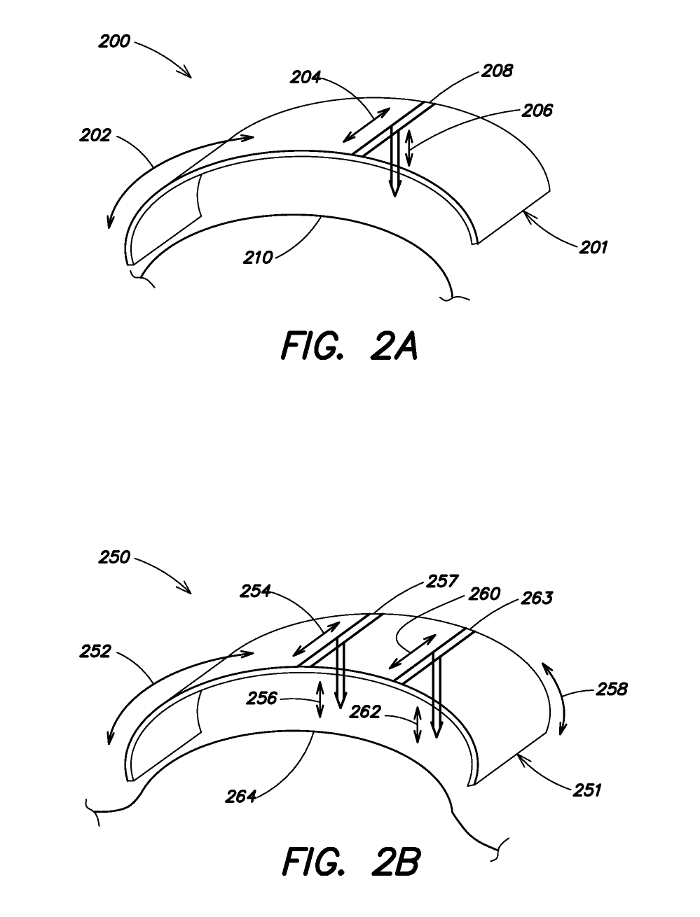

FIG. 2A is a partially transparent view 200 of a programmable surgical device 201. The view 200 illustrates a curved X axis 202, Y axis 204, and Z axis 206 through which a surgical actuator 208 can be position with respect to a body part surface or organ surface (e.g., 210). The surgical device can be constructed to have a curvature that confirms to various body surfaces and/or organs. According to some embodiments, the programmable surgical device is constructed and arranged with a flat configuration for a respective surgical field. Other embodiments can be configured with surgical fields that conform to various contours of body parts and/or organs. Other embodiments of the surgical device can include additional surgical actuators having respective axes of operation.

FIG. 2B is a partially transparent view 250 of a programmable surgical device 251. The view 200 illustrates a curved X axis 252, Y axis 254, and Z axis 256 of surgical actuator 257 and respective X axis 258, Y axis 260, and Z axis 262 of surgical actuator 263. The paired surgical actuators 257 and 263 can be positioned with respect to a body part surface or organ surface (e.g., 264) to perform a variety of surgical procedures. Other embodiments can include additional actuators having respective surgical devices. In some examples, each actuator can be associated with a respective surgical tool and the paired actuators can exchange places to provide multiple surgical tools rapidly to one or more positions during a surgical procedure.

According to one embodiment, the surgical device or surgical system is configured to identify a topography of a target surgical field or the target working surface for the device. In one example, the working surface topography is calculated by various scanning techniques, which can preferably include laser surface scanning but can also include optic scanning, ultrasound, infrared, ultraviolet, or any combination of the foregoing. The system can be affixed to the body part or organ by anchors (including straps, adjustable bands, anchors embedded in tissue, etc.). In some embodiments, the system can utilize tracking point(s) for calculating position in case of movement or maladjustment. In one example, a target surgical field can be demarcated by landmarks prior to affixing the surgical device. In one example, the scanning elements can determine position based on relative distance to the landmarks or tracking points. The device can be programmed to correct for changes in position by correcting x, y, and z co-ordinates associated with actions to be executed.

In some embodiments, the system uses a tool changing protocol to perform working stages of surgery. Further, the system can calibrate a working surface or the target surgical field by re-scanning the topography after each surgical instruction is executed. For example, any changes made during moving, cutting, stapling, suturing or any manipulation of the prior scanned surface can be detected by re-scanning after execution of instructions.

In some examples, the system uses 3-D data of target body organs and/or body parts obtained by magnetic resonance imaging (“MRI”), CT, ultrasound (“US”) and/or optical imaging or a combination of the preceding imaging techniques. In one example, the 3-D image data and/or file can be used to perform simulation of a pre-programmed surgery to confirm the program prior to execution. Once the program is confirmed, the program can be used to operate.

According to some aspects, currently known surgical tools can be constructed and arranged to fit within the surgical actuator of the programmable surgical devices. In some examples, known surgical tools are modified in size and function for use in the surgical devices disclosed.

As discussed, the actuator of the surgical devices can execute surgical programs alone or in conjunction with another actuator. For example, each actuator can be programmed to execute on the same working surface to perform complex surgical tasks.

The size and shape of the programmable surgical device can be constructed and/or varied according to a target surgical area or target body part. The contours of the surgical device and in particular the working surface defined by the lower contours of the device can be designed to perform procedures that range from very small size working surface (e.g., in small organs, including the eye) to larger sized working surfaces. According to some embodiments, smaller size surgical devices can include magnification for performing surgical procedures. In one example, smaller sized systems use magnification or microscopic lenses/magnification to perform surgical procedures.

The X-axis of the surgical module or device can be straight or curved depending on the working surface. The Y-axis can be straight or curved as well. Both X and Y axes can be actuated by small step motors capable of moving in very small steps. In other examples, the surgical actuator can be connected via wires to motors that provide for fine control over movement of the surgical actuator in the x and y axes, as well as in the z-axis.

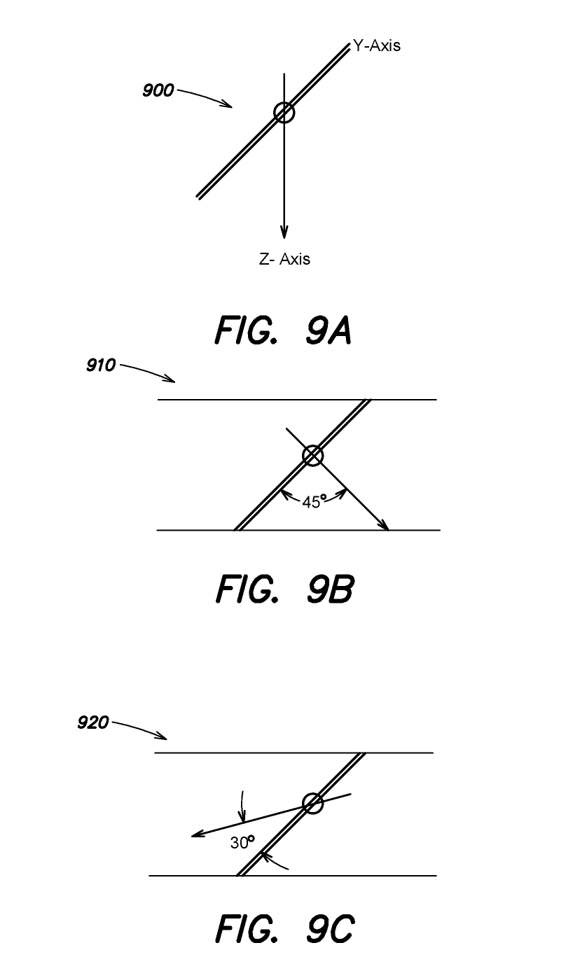

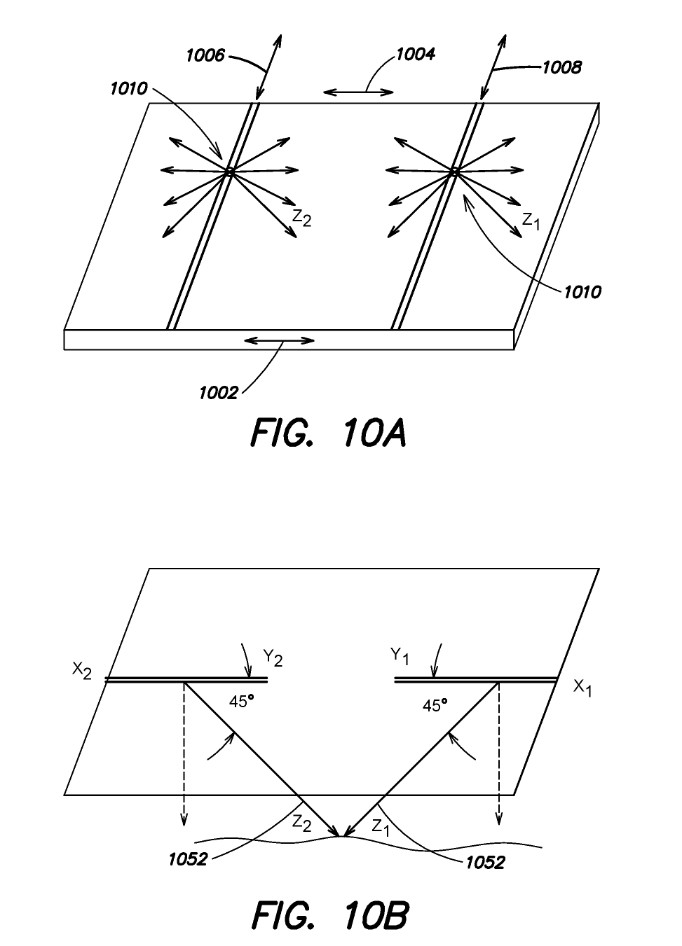

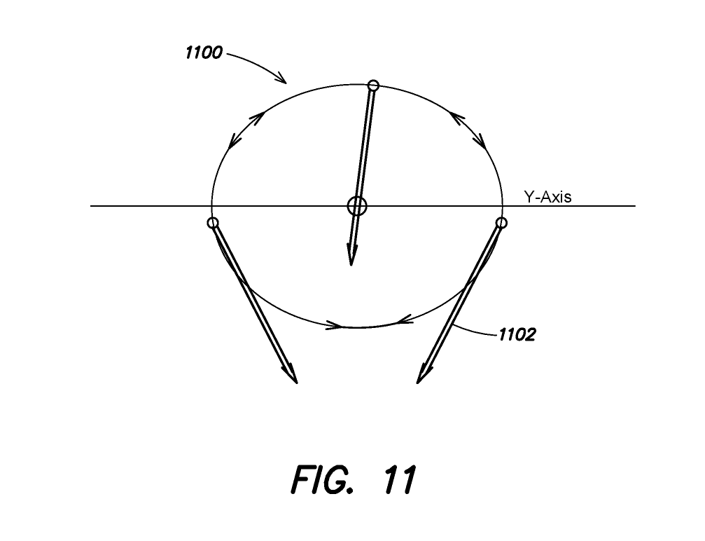

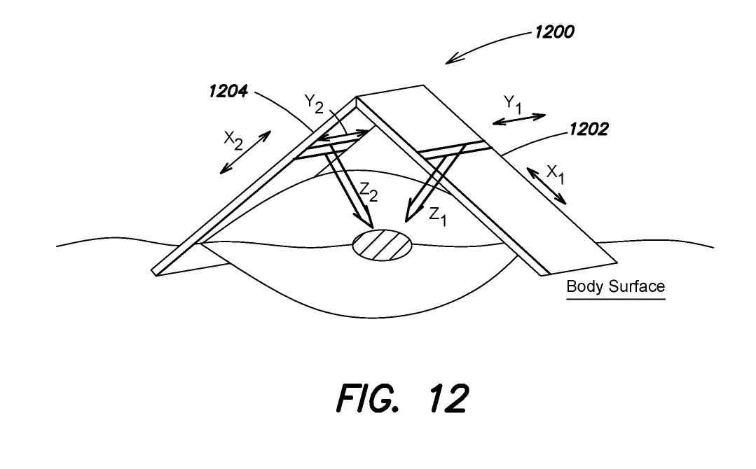

In various embodiments, the X and Y axes can be rigid or semi rigid to conform to a body part or surface. In further embodiments, the Z axis is configured to move up and down in almost 0-90 degrees of freedom in all directions around a target surgical field (360° of rotational freedom). FIGS. 9-11 illustrate example embodiments having Z axes that are operable in various positions. FIG. 9A is a partially transparent view of an embodiment of a programmable surgical device 900 with a Y axis and an X axis having perpendicular positions. FIG. 9B is a partially transparent view of an embodiment of a programmable surgical device 910 with a Z axis configured in a 45 degree Z axis position. FIG. 9C is a partially transparent view of an embodiment of a programmable surgical device 920 configured in a 30 degree Z-axis position. FIG. 10A is a partially transparent view of an embodiment of a programmable surgical device 1000 with multiple axes (e.g., 1002 & 1004 x-axes; and 1006 & 1008 y-axes) and multiple angulated Z-axes 1010 configured to reach target organs and/or body parts. FIG. 10B is a partially transparent view of an embodiment of a programmable surgical device 1050 with multiple axes and the Z-axes 1052 configured at a 45 degree angle. FIG. 11 is a partially transparent view of an embodiment of a programmable surgical device 1100 with a surgical actuator having a Z axis 1102 configured for circular movement with 360 degrees of freedom on the horizontal level and 0-90 degrees of freedom on the vertical level.

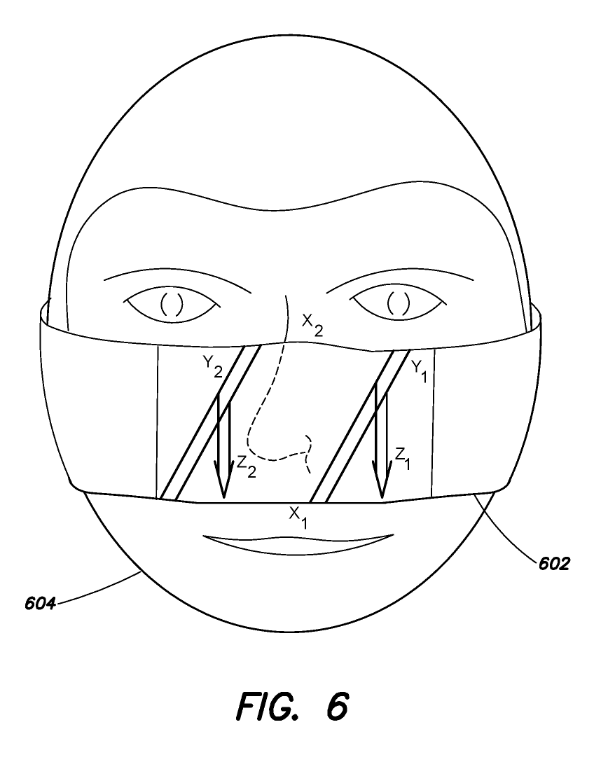

According to various aspects, the surgical system can execute precise surgical procedures to produce pre-programmed shapes or configurations/results (e.g., nose shape manipulation in cosmetic surgery). Shown in FIG. 6 is an example embodiment of a surgical device 602. FIG. 6 is a partially transparent view of a programmable surgical module 602 affixed to a patient's face 604 to perform nose shape change (e.g., cosmetic surgery on the patient's nose) according to a pre-programmed protocol. The surgical program can be executed based on 3D imaging of the patient's nose. The programmable surgical device can include dual surgical actuator system control on respective axes. In some embodiments, the programmable surgical device can include respective curved X-axes and curved Y-axes to facilitate surgical procedures on the patient's nose. In some examples, the surgical device can be rigid with soft base to conform to face topography or the whole device can be semi-rigid.

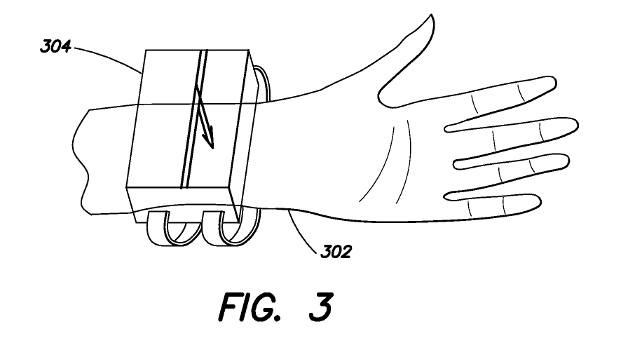

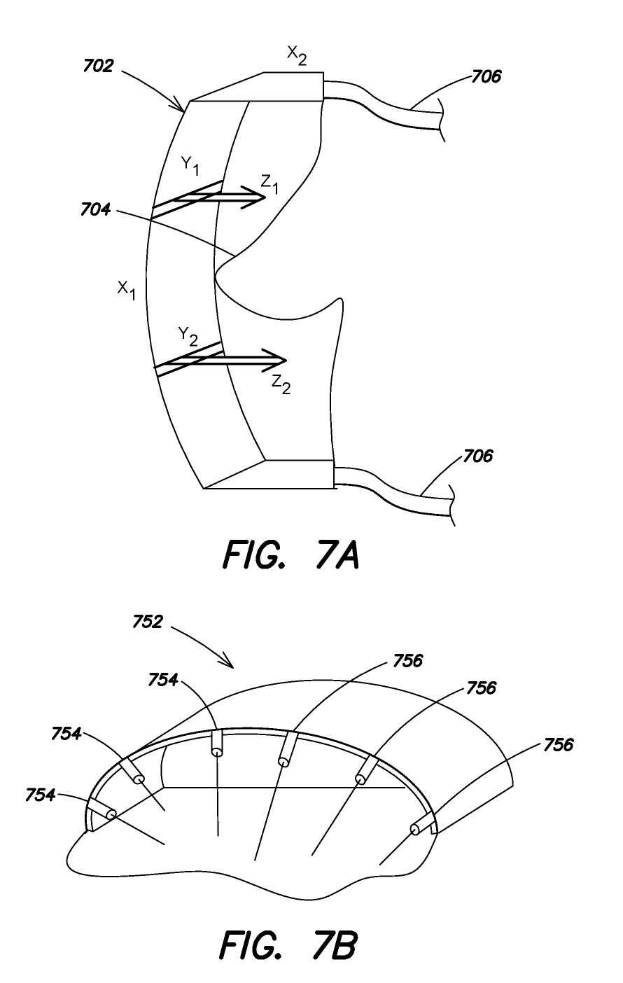

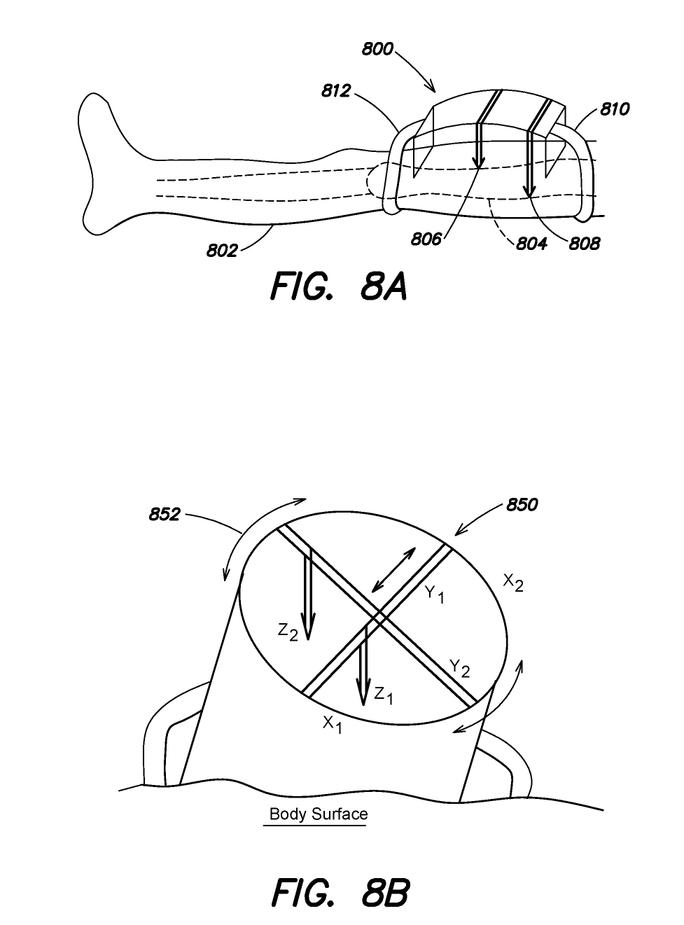

According to various embodiments, the surgical device can be shallow or deep to conform to target body parts and/or organs at a working surface. For example, the device can be almost flat in case of semi-flat body surfaces (e.g., forearm 302 shown in FIG. 3) or it can be conical to operate on odd shapes (e.g., patient's nose shown in FIG. 6 or a patient's breast shown in FIG. 7A). FIG. 7A shows an example device 702 constructed and arranged to conform to a breast shape 704. In some embodiments, the device 702 can be anchored to a patient by straps 706. The device 702 can be configured for reduction, augmentation, and/or reshaping surgical procedures. Returning to FIG. 3 shown is a partially transparent view of a programmable surgical module 304 fixed to a patient's forearm. According to some embodiments, the surgical module can include an open bottom to access a target surgical field. In one example, the working surface for the surgical module is the area of the body surface covered by the open bottom. As discussed above, the surgical module can include scanner components which are used to topographically map the patient's forearm. In one example, the surgical module calculates a depth of the Z-axis responsive to scanning the topography of the target surgical field. Further, the calculations of the depth for the Z-axis can be specific to various areas within the surgical field, and in some examples can be calculated and/or confirmed at each surgical instruction execution. Shown in the FIG. 3, the surgical module 304 can be fixed to body part by anchoring elements or other affixing means to prevent movement or sliding of module over a target surface. In one example, bands or straps are used to secure the surgical module to the patient's arm.

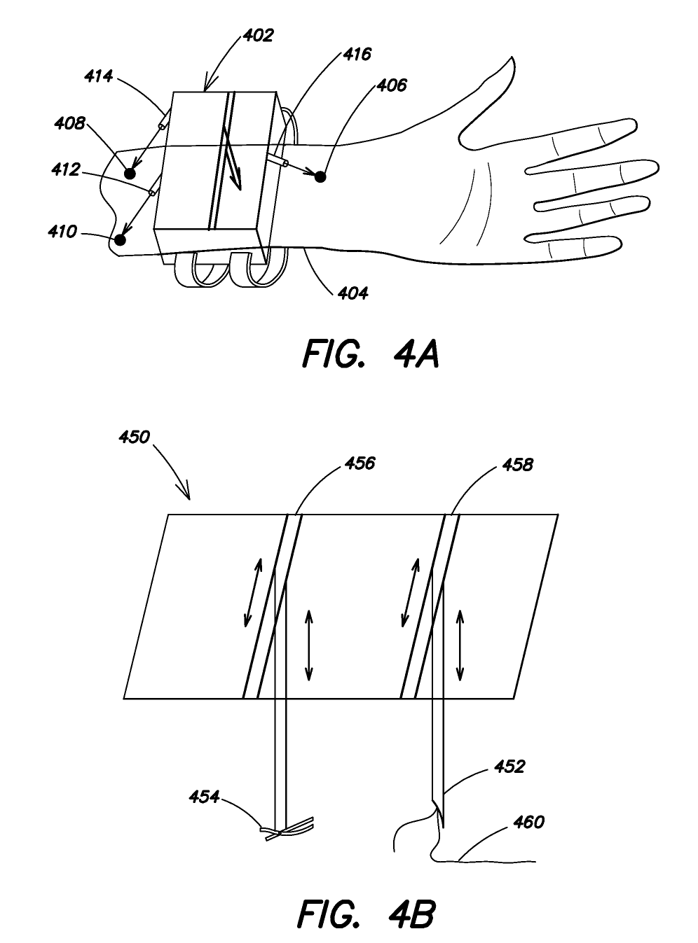

FIG. 4A is a partially transparent view of a programmable surgical module 402 fixed to a patient's forearm 404. Shown in FIG. 4A are three tracking areas (406, 408, and 410) around a surgical field. The tracking areas around the surgical field are used by the system to triangulate position and accommodate any shift in position, for example, during a surgical procedure executed by the surgical module. According to some embodiments, the surgical module includes scanning elements (e.g. 412, 414, and 416) configured to locate and determine position from the tracking areas. The surgical module detects any change in position and uses calculations of position to recalibrate the module. For example, the tracking areas 406-410 can be used to determined position in case of module movement, sliding or changing position, including re-installation of the module. Maintaining positioning information can be used to continue a surgical procedure even where shifting in position occurs. In other examples, the surgical procedures being executed can change the topography of the surgical field.

For example, FIG. 4B is a partially transparent view of a programmable surgical module 450 having multiple surgical instruments (e.g. 452 and 454) on paired surgical actuators (e.g. 456 and 458). For example, the module can include needles 452, holders, clamps 454, suturing needle and thread 460, among other options. During operation, the surgical device can be configured to scan any surgical field for changes in topology. In one example, incisions affect the topology of the surgical field and re-calibration of the device can be required in order to properly close the incision. In other examples, the surgical module/surgical device can confirm position information prior to executing any one or more of the surgical steps in a programmed procedure. In other embodiments, the surgical device can be configured to continuously scan topology and/or measure the surgical field.

According to various embodiments, programmable surgical systems can employ multiple surgical devices/modules. For example, the system can utilize more than one module in the surgical field to produce results via a pre-programmed surgical procedure. According to some embodiments, the surgical field and surgical devices are maintained in a sterile environment from beginning to end of procedure. In further embodiments, an operator can stop and/or start and re-program any surgical procedure. For example, if a new change occurs during procedure, the operator can halt the procedure. Further, the surgical device can detect topology or other physiology that was not part of a 3-D image file of the surgical field and halt any procedure. In various embodiments, the surgical devices can provide suction, irrigation, cauterization, laser welding, sensing, ablation, etc., to complete programmed tasks. In some example, each function can be provided as a surgical tool delivered by a surgical actuator. In other examples, suction, irrigation, cauterization, laser welding, sensing, ablation, etc., can be provided in addition to the surgical tools provided on the surgical actuators.

The programmable surgical devices can be used on a body surface from outside or can be implanted into a body cavity to perform procedures in inaccessible areas or anatomically challenging areas. In some examples, the surgical module(s) can be delivered to a body cavity or lumen manually or by endoscopic, wire-propelled or other delivery mechanisms. In some embodiments, the module(s) maintain close and tight junction with said body part or surface and keeps position by triangulation around surgical field.

In some embodiments, a surgical module can be removed and then re-installed to continue a procedure based on positioning information and, for example, pre-calculated triangulation data from its previous position. Such removal and repositioning can be done in the case of a mal-function or halted procedure. In further examples, the surgical modules can operate on any body material including skin, subcutaneous tissue, bones, cartilage, connective tissue, and parenchyma of organs, among other options. As discussed, the surgical module can use various tools to operate on a variety of tissues and/or positions.

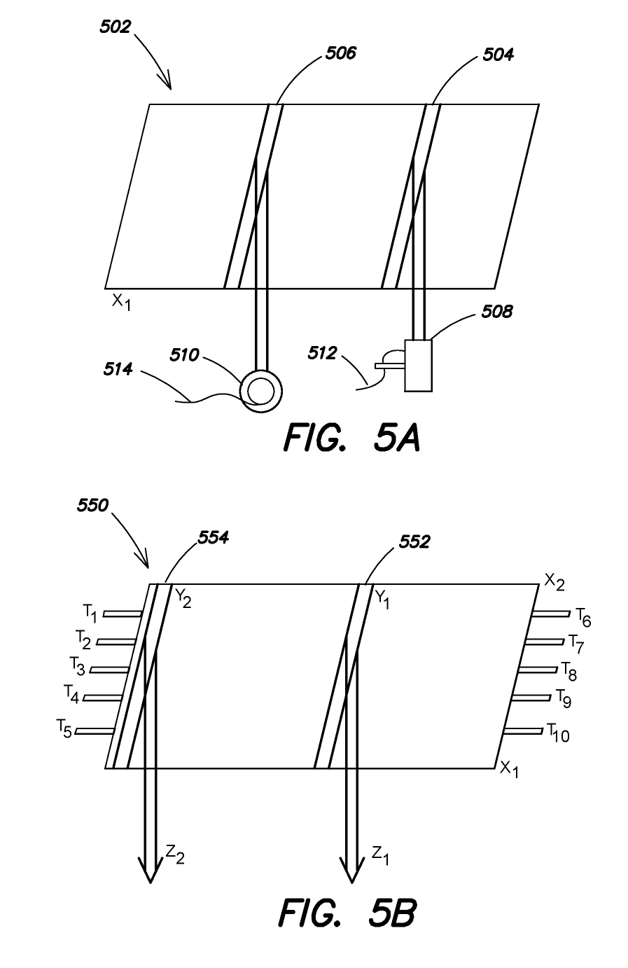

FIG. 5A is a partially transparent view of one embodiment of a surgical device/module 502. The surgical device includes a pair of surgical actuators (e.g., 504 and 506) connected to respective surgical tools (e.g., 508 and 510). The surgical device of FIG. 5A is configured to execute a sewing suture based on operation of the two different threads (512 and 514) suturing across two body surfaces. FIG. 5B is a partially transparent view of one embodiment of a surgical device/module 550. The surgical device shown includes a variety of surgical tools that can be exchanged for a current tool on either actuator. Multiple tools T1, T2, T3, T4, T5, T6, T7, T8, T9, T10, are each available for use by their respective surgical actuator 552 and 554.

The surgical device can also be configured to provide multiple tools in curved embodiments. Additionally, the surgical devices can include multiple scanning members, for example, to ensure correct placement of the surgical tools during a procedure. Shown in FIG. 7B is a partially transparent view of one embodiment of a surgical device 752. Shown are the scanning elements disposed within the surgical device (e.g., at 754 and 756). In some embodiments, the device includes laser scanning elements for determining skin topography. The scanning data can also be used to determine surface calculations, for example, to determine the depth of a Z axis to perform a surgical task.

Shown in FIG. 8A is a partially transparent view of one embodiment of a surgical device 800. The surgical device is positioned to execute procedures on bones 804 within a patient's limb 802. In some embodiments, the surgical device can include multiple surgical actuators (e.g., 806 and 808) having different Z axis geometries. For example, the surgical device can include curved Z axis geometries combined with straight Z-axis geometries. In one embodiment, the device 800 can be fixed in place using straps 810 and 812 or another anchoring mechanism. FIG. 8B is a partially transparent view of one embodiment of a surgical device 850. The surgical device of FIG. 8B includes circular geometries (e.g., 852) for the X axes of respective surgical actuators. The curvature of the X-axis enables complex movements, and complex patterns of movements to execute a surgical program. In one embodiment, the device can include two or more circular x-axes, 2 or more curved y-axes with respect z axes to perform complex movements or curved body surfaces.

As discussed, the various embodiments of the surgical module can perform complex procedures. The procedures can be executed in sensitive organs (e.g., brain, throat, without any effect on surrounding non-surgical parts around a target area). For example, the surgical device can be programmed to remove a brain tumor from a deep brain location by means of pre-programmed pathway that avoids surrounding sensitive brain structures with complete precision.

In some aspects, the system can simulate results for the patient before a procedure. Imaging data can be combined with the 3-D file of the simulated part or organ to produce simulated results with complete precision. Further, the results of the procedure can be simulated to provide precise expectation of results (e.g., cosmetic surgery results can be provide, including nose reduction or breast reduction surgery results).

In some embodiments, the surgical modules can be equipped with CCD chips and other imaging means to give operator a close-up view of surgical field and target area(s). In further embodiments, the surgical modules can be used for delivery of various therapeutics to anatomically challenging areas (e.g., delivery of radiation to a small tumor inside brain tissue or injection of chemotherapeutic agents inside the eye or using laser beam at target areas inside blood vessels for removal of a clot without causing bleeding, among other options).

In some examples, the surgical module is programmed to injecting dye material under the skin to the epidermis to produce complex and minute shapes used in coding and/or data storage.

In other examples, the surgical modules can create 3-D shapes in the epidermis that can be read by a specific decoder and capable of being coded or re-coded according to needs. These 3-D shapes or tattoos store data and can be used to give certain commands or store information about the subject (e.g., providing medical record information). In further examples, the 3-D shapes contain nodes at different depths from the skin surface. Each node can be read only at this depth by certain decoders capable of sending waves to each level of the 3-D structure to read data at said level. In some embodiments, the nodes can carry information at different security levels. The amount of data decrypted from each node depends on the level of clearance of the decoding reader. In further embodiments, each node can be subject specific with all biographic data stored to define identity, characteristics or other information related to subject. It can be used to locate subject by GPS if the nodes can be read remotely (magnetic, wireless, microwave) or other communication protocols between the 3-D code and reader.

According to some embodiment, surgical procedures can be programmed to use multiple surgical devices. FIG. 12 is a partially transparent view of one embodiment of a surgical system 1200. The surgical system includes interlocking surgical devices 1202 and 1204. For example, interlocking surgical devices can be used to provide a surgical platform for accessing curved portions of anatomy. In one example, operations on a patient's eye can be executed by interlocking surgical devices. Show in FIG. 12 is a procedure for intravascular lens change based on contoured surgery Z inter-locking surgical modules. The interlocking modules execute a programmed surgical procedure on the curved body surface (cornea) to perform complex surgical movements around the eye, for example, to change the intra-ocular lens for contact treatment.

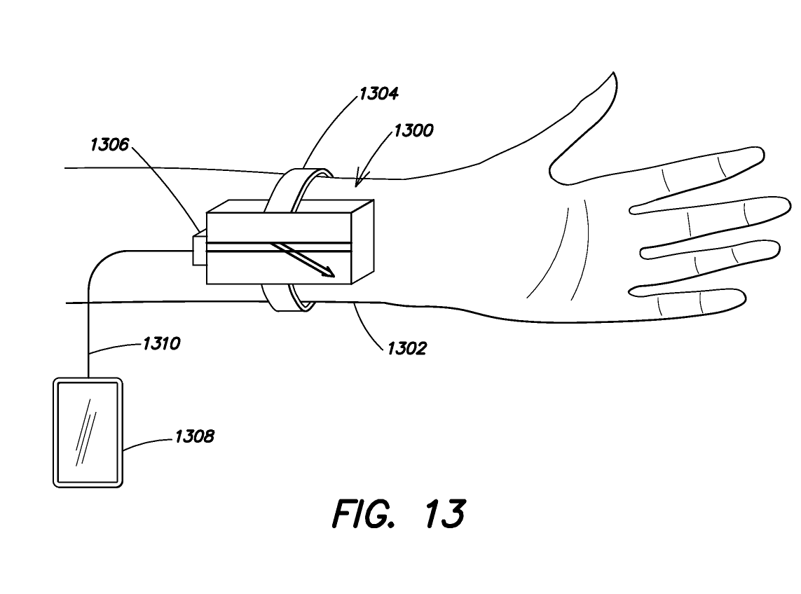

Shown in FIG. 13 is a miniature CNC machine 1300 attachable, for example, to a person's forearm 1302. The device 1300 can be attached via straps 1304. In some embodiments, the device 1300 is configured for use on top of the skin. The device can include ink and/or dye reservoirs for printing an image on the skin. In one embodiment, the device 1300 includes a connection port 1306 (e.g., USB port) to connect to computing devices, including for example, a smart phone 1308 via a USB cable 1310. In some examples, the connection port can be physical or can include wireless communication components.

According to one embodiment, the device is configured to receive an image from the connected computing device. The device 1300 can be configured to translate the image data into program instructions to print the image on skin. In some embodiments, the device 1300 is configured to retrieve brush tools that are connected to ink and/or dye reservoirs. The device then prints the image. In other embodiments, the device 1300 can include tattooing needles and ink reservoirs for tattooing the image into the skin. According to other embodiments, the user of the computing device can download an application. The application can be configured to translate image data into CNC instructions for printing the image data. In further embodiments, the device is configured to map the person's forearm and adapt the CNC instructions to the topology of the person's forearm.

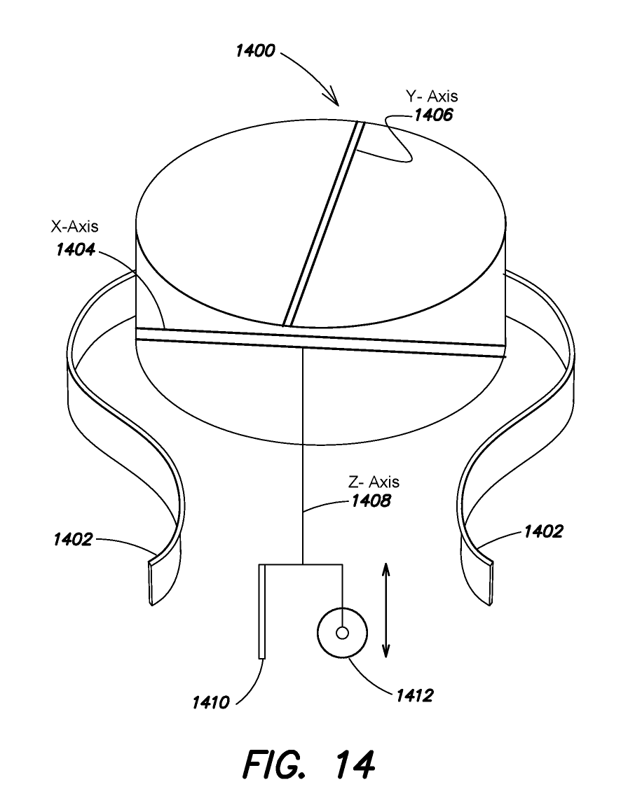

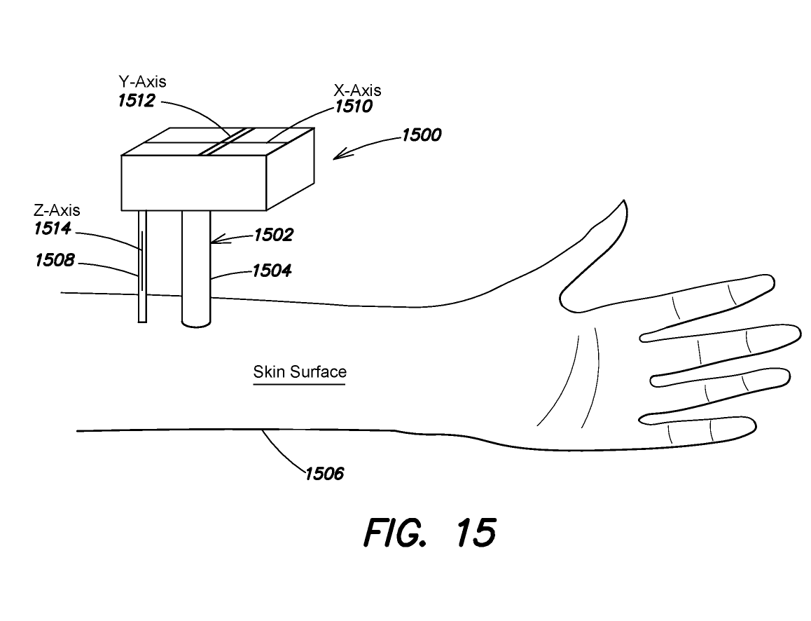

According to another embodiment, the miniature CNC machine can include a print head. Shown in FIG. 14 is a programmable surgical device 1400 that includes a strap 1402 (e.g., Velcro strap) for anchoring the device to a person and/or body part. The device 1400 can include a print head 1410 moveable along x, y, and z axes (1404, 1406, and 1408). In some examples, the print head can include inkjet print heads, micro-pin print heads, etc. In some embodiments, the device 1400 can include a lead wheel 1412 for mapping the topography of surface to be printed (e.g., skin or a person's forearm). The device is configured to manage printing with the print head based on using the lead wheel 1412 as a reference point for executing printing instructions. In other embodiments, the device uses positioning information for the lead wheel to adjust the CNC instructions for printing responsive to the positioning of the lead wheel 1412.

FIG. 15 is another embodiment of a miniature CNC machine/surgical device 1500. The device 1500 includes at least one laser scanner 1502 for mapping the topography of an operating field (i.e., area to work on) using a laser beam. In one example, the laser 1502 is used to map the topology of a person's forearm 1506. A moveable printing head 1508 can be controlled based on the mapped topology. In some embodiments, the printing head 1508 and/or the laser can be moveable in x (1510), y (1512), and z (1514) axes.

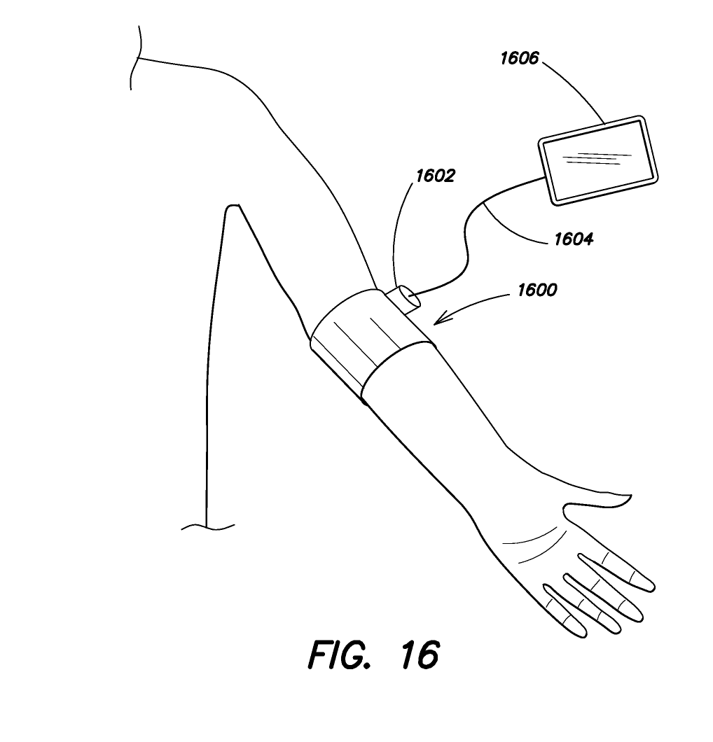

FIG. 16 illustrates another embodiment of a miniature CNC machine 1600. The device 1600 is constructed and arranged to extend around a body part or extremity on which a procedure can be performed. In some embodiments, the device 1600 is constructed as a cylindrical body or a semi-circular body that can be fixed to a body part with one or more belts and/or Velcro straps. The device can include a communication port 1602 that can provide for a physical connection via a USB wire to a computing device (e.g., smart phone 1606). In some alternatives, the device 1600 can be equipped with a wireless communication port.

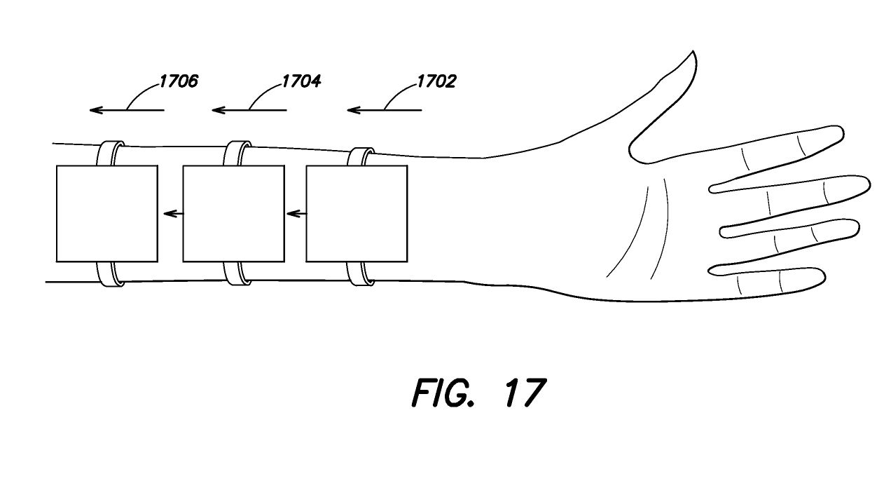

FIG. 17 illustrates another embodiment of a device 1700. The device 1700 is configured to print a large size image, for example, on the whole forearm by moving it to cover the whole print area. In some embodiments, the device is configured to scan the initial print surface to determine its position, print a portion of the image, and re-determine position in relation to the whole image upon being moved along the print surface. The device 1700 can then print a next section of the image until the image is complete. In FIG. 17, device 1700 is positioned at at least three positions 1702, 1704, and 1706 to produce a complete image. In some embodiments, the device 1700 is configured to print temporary reference points on the person's skin, so that the device can obtain accurate positioning information in order to continue printing.

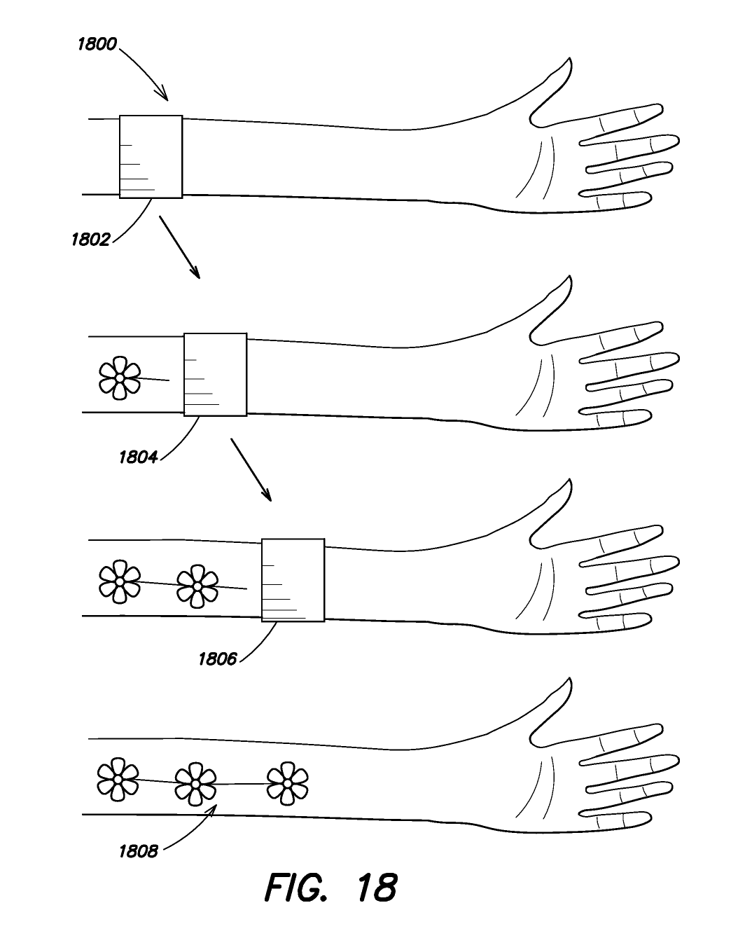

FIG. 18 shows an example printing including repositioning of the device 1800. The device is moved along a print surface from position 1802 to 1804 to 1806 until the complete image 1808 is generated. The final complete image is completed by moving device on common image points in-between pics.

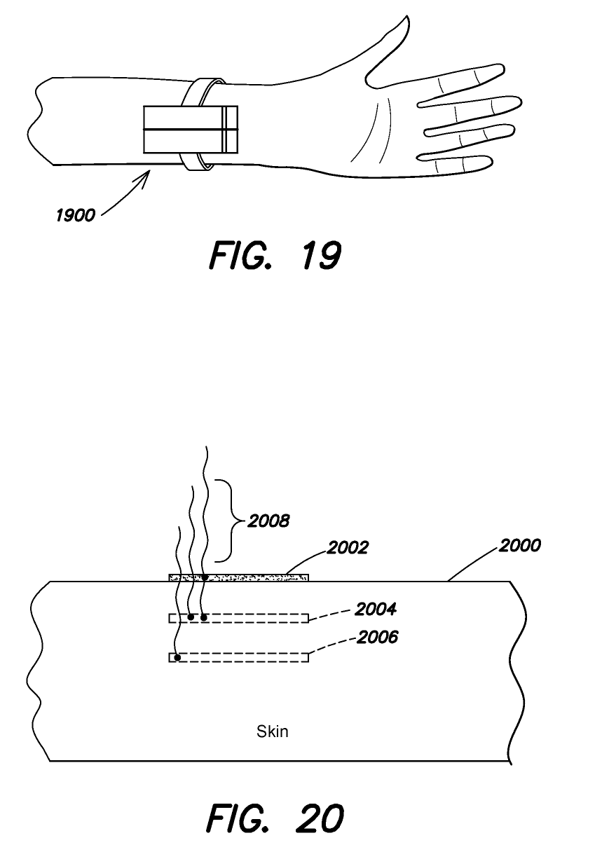

FIG. 19 shows another embodiment of a miniature CNC machine or a programmable surgical device 1900. The device 1900 is configured to print an image on both the skin surface and with a needle to print another image under the skin (e.g., 1 mm under the skin's surface) to create two separate elements of a 3-D image. In other embodiments, the device is configured to generate 3-D renderings that extend through multiple levels and/or multiple depths below the skin's surface to generate complex 3-D images. In some embodiments, the generated 3-D image can only be read by laser beam having a specific wave length. In other embodiments, different imaging components can be required to read or process the subcutaneous portions of the image. In further embodiments, surface images can be generated in conjunction with subcutaneous images. As discussed above, information can be encoded in the images. Further, the 3-D images or tattoos and/or subcutaneous components of the images can be used as authentication or computer security measures. In some implementations, the surface image is presented as a portion of a security measure that must be read in conjunction with subcutaneous images to meet the security requirements.

FIG. 20 shows an example of a surface and a subcutaneous image according to one embodiment. The person's skin in the relevant area is illustrates at 2000. A surface portion 2002 of an image has been generated (e.g., by a miniature CNC machine/programmable surgical device). Other portions of the image have been generated subcutaneously at 2004 and 2006. In some embodiments, the subcutaneous portions of the image (e.g., 2004 and 2006) are generated such that a laser having specific wavelengths is required to read the whole image. In further embodiments, each portion (e.g., 2004 and 2006) can require a corresponding wavelength of light. In yet other embodiments, a single laser and wavelength can be used to capture the subcutaneous portions of the image.

In some examples, an image made of two or more layers of coded images under the skin is generated so that the image can be read only by a specified laser beam having a specific wavelength focused at each pre-specified depth. Thus various embodiments are configured to create a 3-D secure code including subcutaneous ink that can be invisible to normal light.

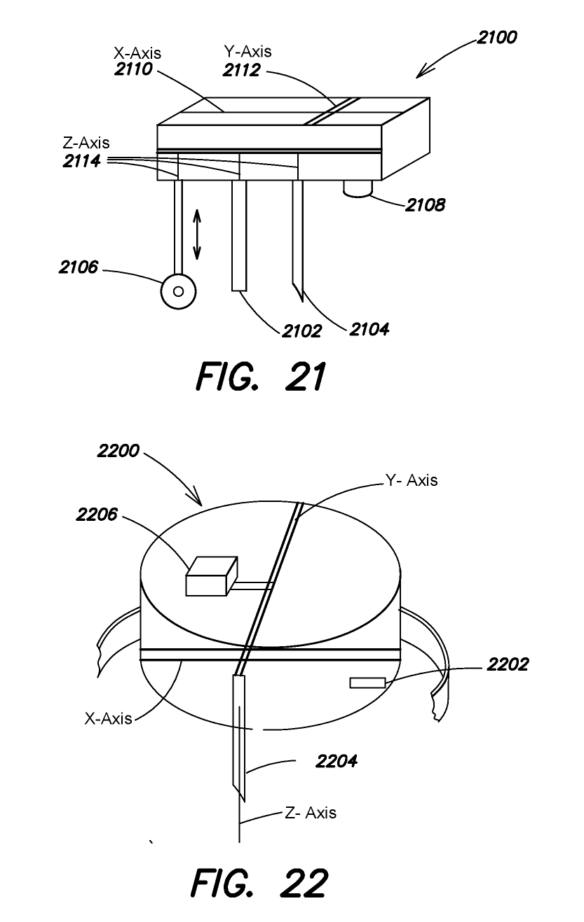

FIG. 21 is another embodiment showing a programmable surgical device 2100 that can be configured with a surface print head 2102 (e.g., inkjet print head) and a needle head 2104 for printing subcutaneously. In some embodiments, the device can include a lead wheel 2106 or a guide wheel. The lead wheel provides a dynamic surface reference for calibrating the device and/or modifying print instructions according to a variable skin surface. The device 2100 can also include laser scanner components 2108 for imaging and/or mapping a topology of a skin surface. Once the surface has been mapped, image printing instructions can be modified to manage variability in the print surface. In one example, a device with an inkjet head and needle head is provided to create 3-D images having image portions at multiple skin layer depths. The surface topography can be determined by either a lead wheel or laser scanner. An either printing implement can be moveable along any of an x-axis 2110, y-axis 2112, and a z-axis 2124.

According to another embodiment, FIG. 22 shows a programmable surgical device with a laser scanner 2202 and high-speed oscillation needle 2204 on z-axis configured to create 2-D or 3-D images under the skin. The device is configured to generate images under the skin based on laser triangulation of surface topography. In some examples, a 3-D image is used as a secure code that can be read by a specific laser beam wavelengths. In further examples, the device 2200 can include invisible ink reservoirs 2206. The inks in the reservoirs can be selected based on being visible only in response to specific frequencies or wavelengths of light.

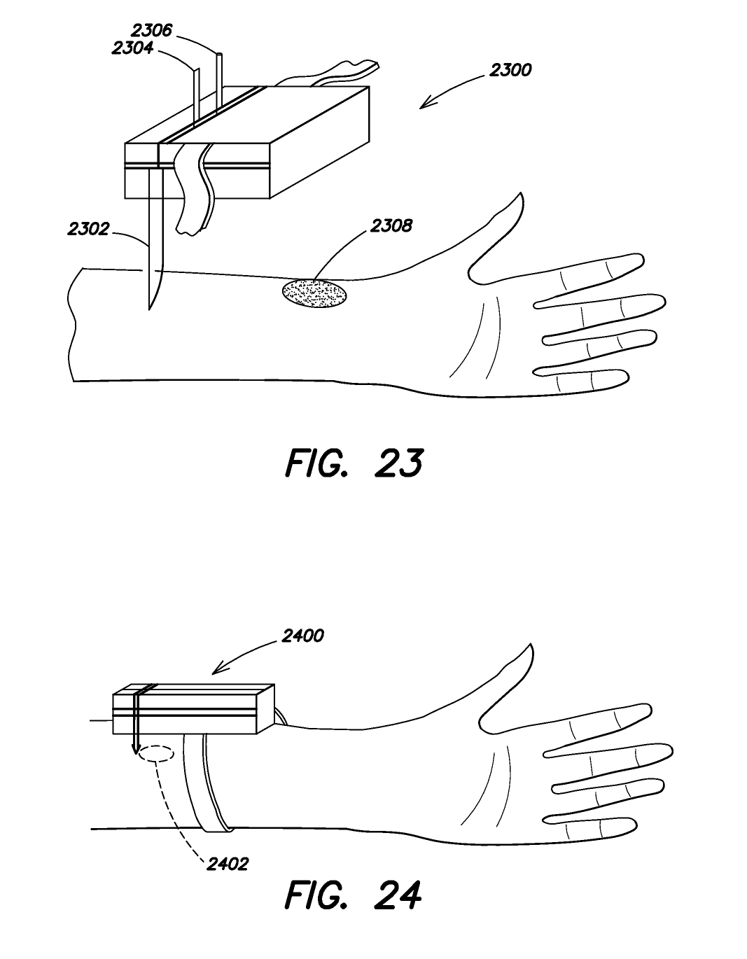

In FIG. 23 is an embodiment of a programmable surgical device 2300. The device 2300 includes exchangeable z-axis heads (e.g., scalpel head 2302, staple head 2304, and cauterizing head 2306, among other examples) to excise a skin lesion 2308, wart, and/or tumor from a skin surface. Imaging of the skin surface is used to identify the skin lesion 2308. The device 2300 then moved a scalpel head 2302 to the lesion, and the lesion is cut away. In some embodiments, any bleeding can be stopped by cautery, and cautery can be followed by wound closure (e.g., via stapling or suture).

FIG. 24 shows another embodiment of a programmable surgical device 2400 and a tumor 2402 that is the surgical target of the device. In one example, the device is configured for excising a deeper skin lesion from under the skin surface.

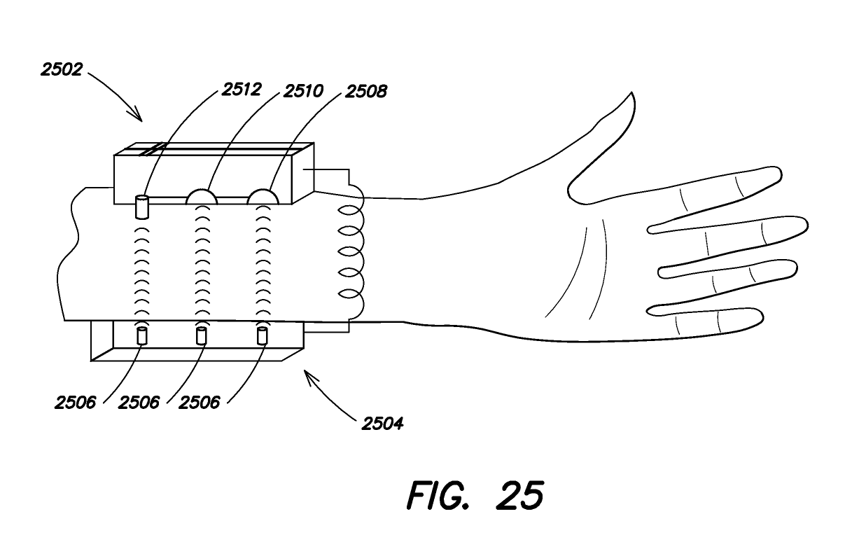

FIG. 25 illustrates another embodiment where more than one surgical device (e.g., 2502 and 2504) is attached to a patient. The first 2502 and second 2504 surgical devices operate in conjunction to facilitate improved imaging and execution of a surgical procedure. According to one embodiment the 2^{nd} device 2506 can be connected to first device 2502 so that each device can locate the other in space to determine a 3-D surgical field, for example, for the first device. In one example, the second device includes a plurality of ultrasound emitters (e.g., at 2506) and can also include laser emitters. In some examples, the first device can include respective receivers for receiving any sound or light waves emitted by the second device. In one embodiment, each emitter uses a specific frequency and each respective receiver is configured to each specific frequency. The device 2500 determines a surgical field responsive based on a known distance between receivers and emitters and the distance of each device from each-other. With the known distances and the received signals, the device is configured to calculate a 3-D surgical field of the whole body, and a surgical procedure can be crated and executed to treat deeper maladies, than for example, surface lesions and/or surface tumors. Additionally, tumors having multiple orientations and/or extending over larger areas can be mapped and completely excised using multiple devices.

According to another aspect, a miniature CNC machine or a programmable surgical device is configured to receive digital image information and translate the digital image into instructions for printing a tattoo on a surface of skin of a person. The device can also be configured to calibrate the printing of the tattoo responsive to determining a surface topology and/or responsive to dynamic reference point between the device and the skin surface (e.g., provided by a lead wheel or guide wheel). Various embodiments, of the miniature CNC machine or surgical device can provide any single one of the following elements, any multiple ones of the following elements, and any combination of any two, three, four, five, six, seven, eight, nine, ten, or more of selections within following elements (up to an including selections of all of the following elements):
Translate an image can from any picture (e.g., gif, .jpg, .png, .pdf, etc.) from a computing device (e.g., smart phone or digital camera);
a connection between the image source (e.g., computing device) and the surgical device can be wired or wireless (e.g., wi-fi, Lan, Wan, Bluetooth);
includes a printing head moveable in x-y-z axes, responsive to CNC instructions or other instructions defining a x co-ordinate, y co-ordinate, z-coordinate and an action;
surgical device can include an x-axis and y-axis having a curved path, which can be contoured to follow the surface topography of a print area;
the z-axis moves up and down carrying, for example, a printing head based on surface topography;
surface topography and or a dynamic surface reference can be established by a mechanical lead wheel that follows all curves of print area and followed by the print head moving up and down, and can be manipulated responsive to the movement of lead wheel;
the surgical device can include multiple emitters and/or receivers to generate a surface topography (e.g., topography can be depicted by multiple frequent laser scans of surface) which controls the movement of the z-axis. In one example, the surface topography is the working space of z-axis and is assigned to a zero value regardless of how it's shaped;
the printed image can be actual pic or bar code or other code format;
the ink can be monochrome, poly chrome, invisible, or any combination, and some embodiments can include multiple ink reservoirs;
the ink is temporary, semi-permanent, or permanent;
the image can be much larger than the actual size of device, and for example, can be drawn by moving the device over a large print area (for example, the device can read the part of image printed and add to it until full image is complete);
the device can be affixed to print area by belts, Velcro, or other anchoring mechanism;
configured to receive an image or code and print it on skin surface or body part after determining the topography of said part using both a print head and a tattooing high frequency needle;
the print head creates the surface over-skin image;
the needle can be configured to create the under-the-skin image in another one or more layers at various depths;
configured to defined multiple subsurface image layers, for example, each layer has its own specific depth and own ink that can be read by a specific laser beam wavelength at said depth;
the subcutaneous images can be made by visible, invisible, or digital ink;
digital ink can create a dynamic pic that can be preprogrammed or changed by electrical magnetic or optic control;
the needle head can create both the skin image and subcutaneous image;
the print head can print a covering image to obscure the underlying deeper image which can be only read by certain wavelengths;
pre-programmable to execute a surgical procedure by manipulating one or more through one or more z-axes;
execute surgical procedures on a skin surface (e.g., removal of a wart or biopsy);
execute complex surgical procedures involving deeper layers of tissues and imaging based direction/confirmation of surgical procedure;
determining a 3-D surgical field using multiple surgical devices and creating a surgical program responsive to the 3-D surgical field;
one or more programmable surgical devices configured for at least 5-axes surgery on 3-D structures (e.g. cosmetic surgery on nose or joint replacement or bone surgery); and
one or more programmable surgical devices configured for micro-surgery (e.g., on eye or brain).

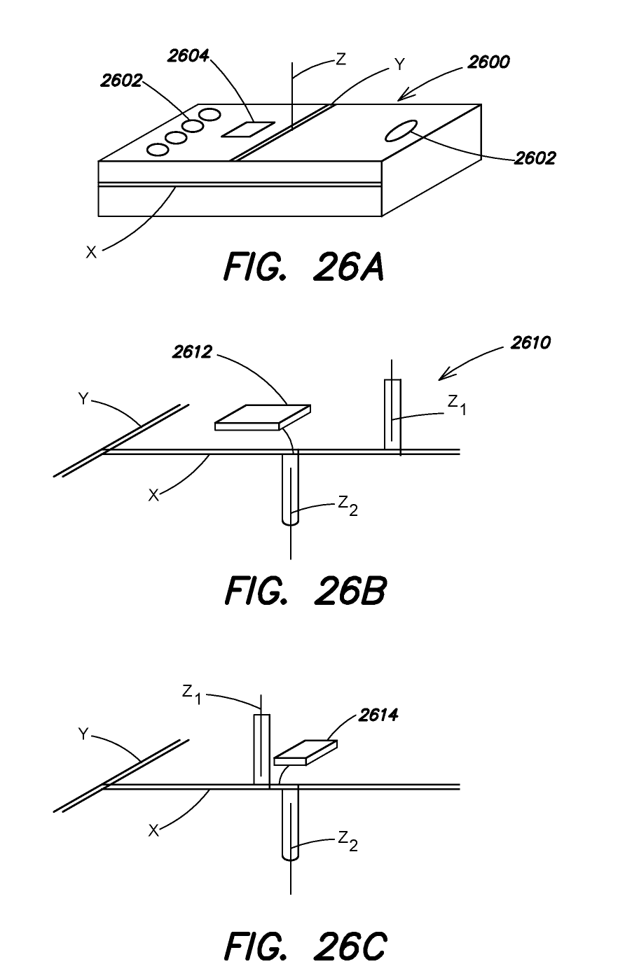

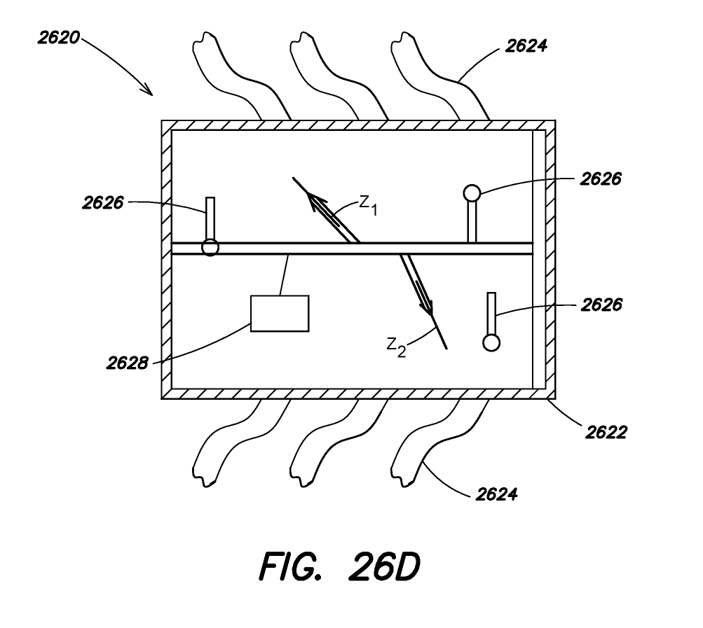

Shown in FIG. 26A-D are views of a programmable surgical device configured for image printing and/or tattooing of images on a skin surface. In FIG. 26 A, a device 2600 is shown from the operating surface down. The device includes a plurality of laser scanning elements configured to triangulate position and track any movement of the device 2600 with respect to a working surface. The device can be configured to modify procedure (e.g., surgical procedure, print procedure, etc.) instruction responsive to any detected movement. In FIG. 26B shown are internal structures of a device 2610 showing a first tool operable along a first axis (Z1) and a second tool operable along a second axis (Z2). Both tools are position able along the X and Y axes. In FIG. 26B, the ink reservoir 2612 is connected to the tool operating at the Z2 position.

In FIG. 26C, shown is another configuration where a common ink reservoir 2614 is connected to both tools shown at positions Z1 and Z2. In other embodiments, one or more ink reservoirs can provider ink, dye, etc., to any number of tools in a surgical device. FIG. 26D shows a surgical device 2620 from a side view. The bottom circumference 2622 of the device 2620 can be constructed and arranged of a semi-rigid material so that the bottom circumference of the device 2620 can conform to a body part or surface when attached. For example, straps 2624 can be used to anchor the device 2620 to a body part or surface and the bottom circumference 2622 can conform to the body part or surface. The device can include laser scanners (e.g., at 2626) to provide topography and/or positioning information to the device 2620. In some embodiments, the device includes ink or dye reservoirs (e.g., 2628) for printing images on skin or tattooing images on and/or under the skin.

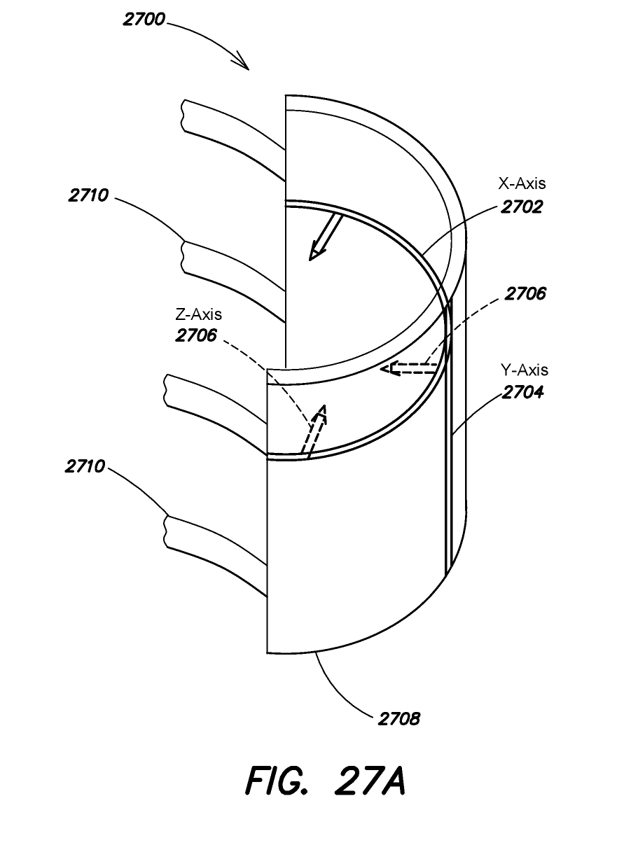

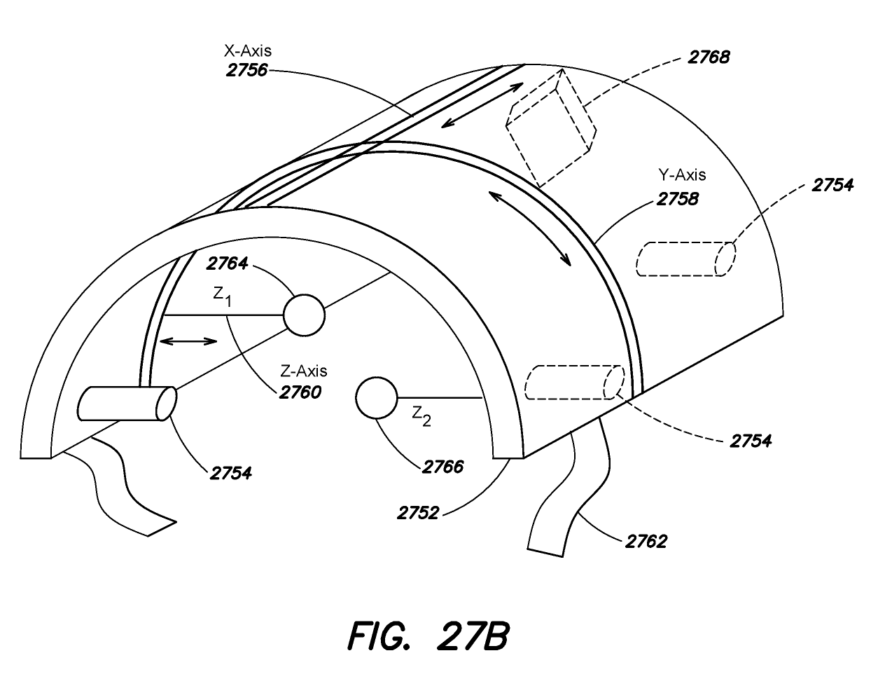

FIG. 27A is another embodiment of a surgical device 2700. The body of the surgical device 2700 is constructed to be semi-cylindrical. The x-axis 2702 follows the curvature of the cylindrical portion, and the y-axis 2704 travels along the length of the body. As shown, surgical tools, print heads, lead wheel, etc. can operate along the z-axis which is directed towards the interior of the cylindrical portion 2708. As is other embodiments, straps 2710 can be used to connect the device to a body part (e.g., arm, forearm, leg, foot, etc.). In FIG. 27B shown is another semi-cylindrical embodiment of a surgical device 2750. Laser scanners (e.g., 2754) are positioned to direct light energy towards the interior of the cylindrical body 2752 to map an operating surface. The moveable surgical tools are configured to travel along multiple axes (e.g., x-axis 2756, y-axis 2758, and z-axis 2760). In some embodiments, the assignment of the x and y axes can depend on the orientation of the device when attached (for example, with straps 2762. In some embodiments, surgical tools are configured to perform operations along the z-axis (e.g., at positions Z1 and Z2). In further embodiments, each tool can be paired with a physical guide or a guide wheel (e.g. 2764 and 2766). The guide wheel can be extended until contact with an operating surface. The surgical tool is paired with the guide wheel such that the guide wheel sets a zero depth for the z axis that will follow the contours of the operating surface regardless of curvature. In other embodiments, a laser scanning head can be used in place of the guide wheel and the laser scanning head used to establish zero z axis position that conforms to the operating surface (e.g., detects contours, channels, bumps, hills, valleys, etc.). Shown in FIG. 27B is an optional ink reservoir 2768 for painting and/or tattooing images onto an operating surface.

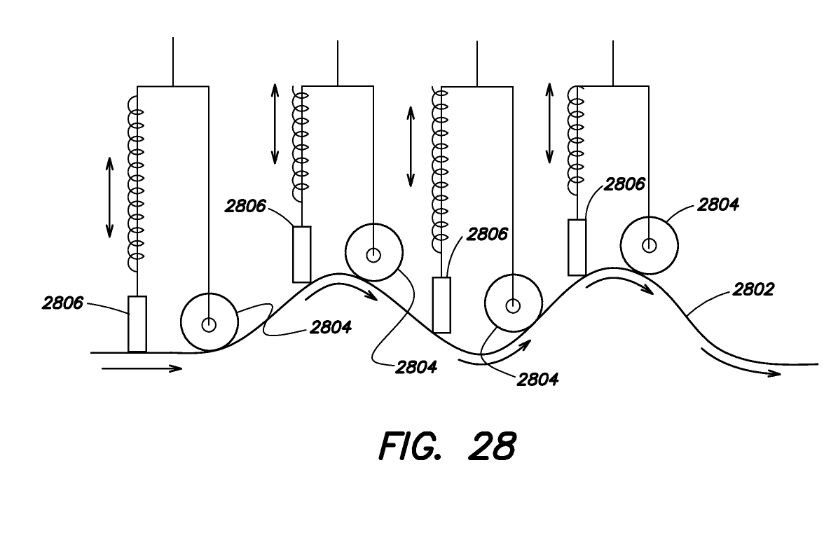

FIG. 28 illustrates the operation of a guide wheel or lead wheel 2806 paired with a surgical tool, print head, etc. (e.g., 2806). The guide wheel 2804 is configured to move up and down a z-axis responsive to features of the operating surface 2802. As the guide wheel 2804 encounters hills, valleys, and/or other features, the guide wheel is configured to extend or retract accordingly. In this manner, surgical or printing instructions that employ depth information can be executed on a variable surface accurately and with precision. According to some embodiments, the guide wheel is used to discover changes in topography over a surgical field or operating area. The deployment of the guide wheel can be used by the device to establish a zero position or reference in the z-axis. In some examples, setting the dynamic position of the guide wheel to zero enables the device to implement depth based procedures without adjusting an original program and/or to use the original program regardless of variation between operating surfaces.

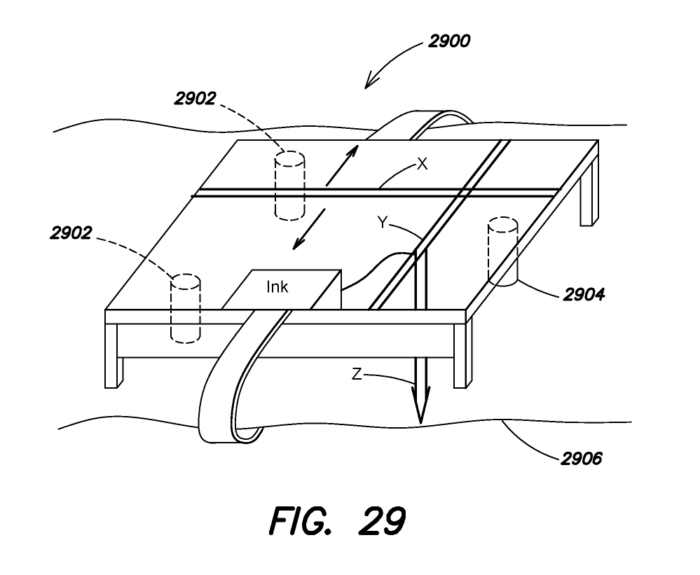

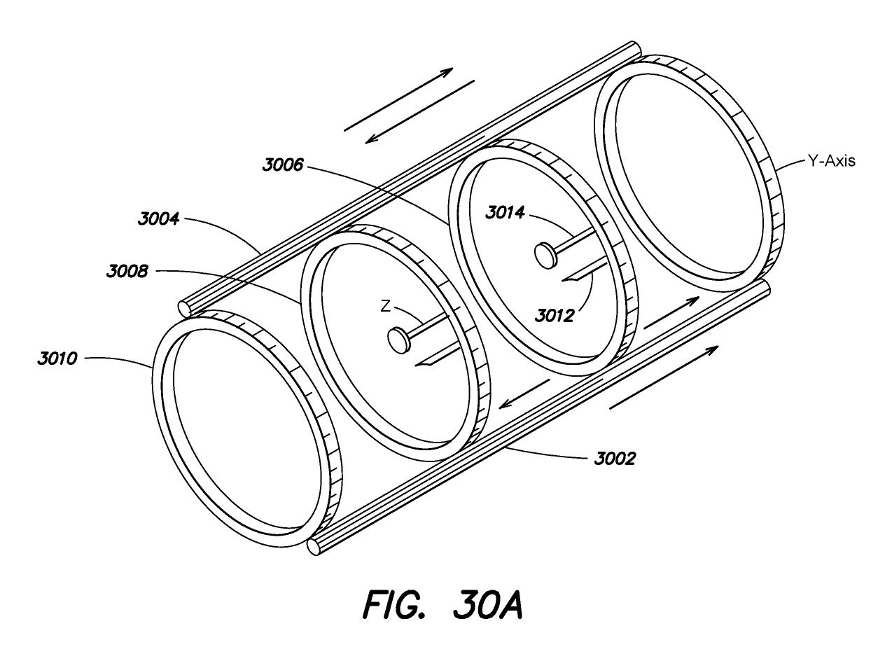

FIG. 29 shows another embodiment of a surgical device 2900. Device 2900 is constructed and arranged to provide a raised platform over the operating surface. In some examples, a raise platform is implemented to facilitate laser scanning of the operating surface. For example, lasers shown at 2902 and 2904 can be positioned to facilitate scanning of the operating surface before, after, and during any procedure. In some embodiments, laser scanning is continuously performed during a procedure. Execution instruction can be modified responsive to any changes in surface topography (e.g., skin surface 2906).

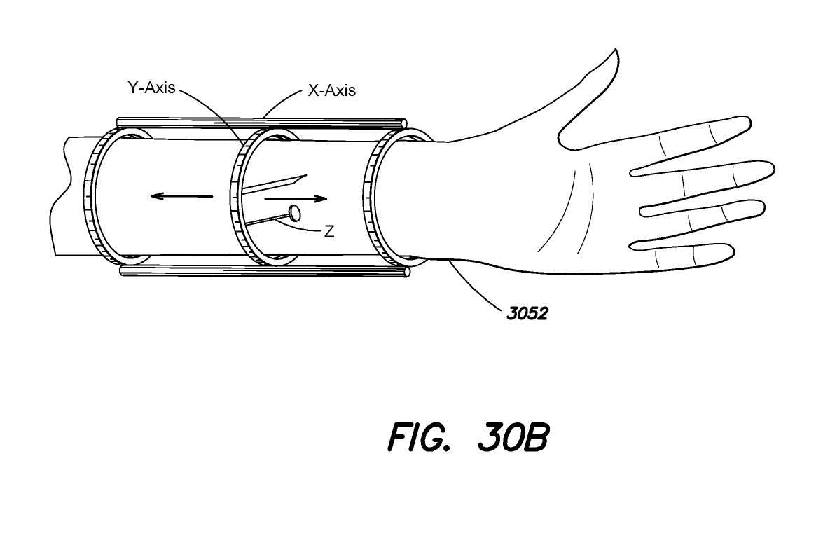

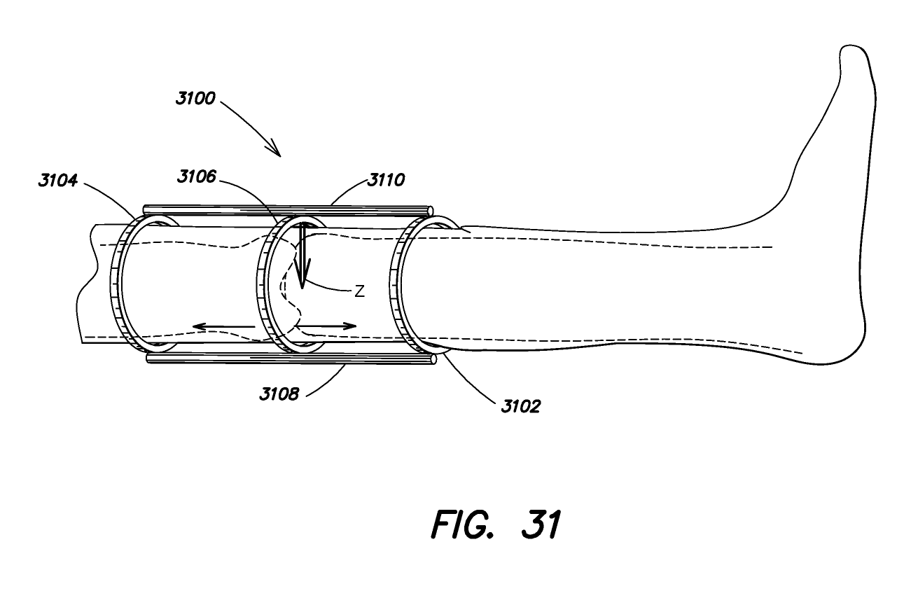

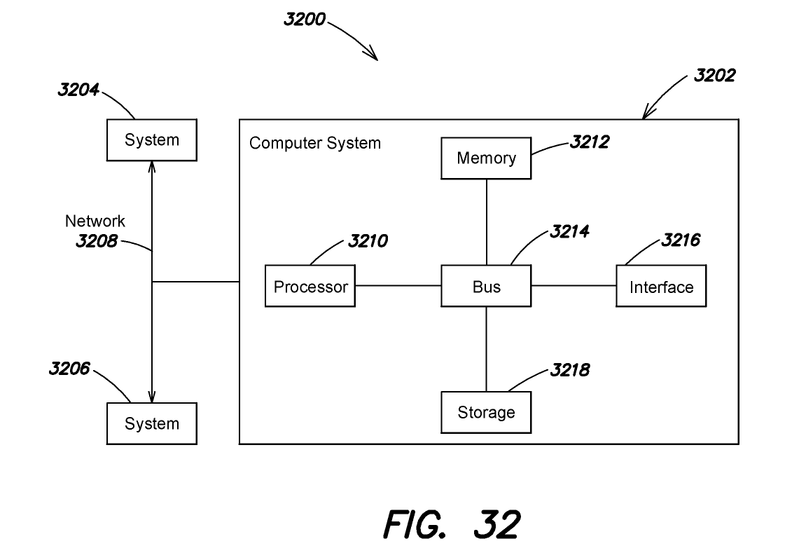

FIG. 30A shows another embodiment of a surgical device 3000. The device 3000 is constructed and arranged to form a sleeve that fits over a body part or surface (e.g., a patient's forearm). The device 3000 can include one or more threaded bars (e.g., 3002 and 3004). One or more threaded rings (e.g., 3006, 3008, and 3010) can be attached to motors on the one or more threaded bars. By operation of the motors the threaded rings can be position along the x-axis defined by the device 3000. One or more surgical tools, print heads, etc. can be attached to each threaded ring. For example, a surgical tool attached to the threaded ring can be position anywhere on the threaded ring (i.e., moveable along a y-axis). Each surgical tool can then be programmed to operate along a z-axis, for example, extending towards the interior of the sleeve. In one example, based on a z-axis value the surgical tool can cut to a specified depth excise tumorous tissue, etc. In other embodiments, the device can execute a program to suture closed an incision, cauterize an incision, among other options. In other embodiments, the rigid rings can be fixed in place, the surgical tools disposed on the threaded bars so that the surgical tools are position able according to an x, y co-ordinate. A z value can specify a depth at which the surgical device 3000 will perform an operation.

According to some embodiments, a tool 3012 (e.g., surgical tool, print head, high frequency oscillation needle) can be paired with a reference guide 3014 (e.g., guide wheel) that maintains a zero depth value by travelling along a variable operating surface. The tool can be deployed based on the zero depth valued determined from the reference guide. FIG. 30B shows another view of a cylindrical embodiment of a surgical device 3050 in place on a patient's forearm 3052. FIG. 31 shows another implementation and placement of a cylindrical surgical device 3100. The threaded rings 3102, 3104, and 3106 (for example, metal threaded rings) can be connected to two or more threaded bars 3108 and 3110 (e.g., threaded metal bars). In some embodiments, the attachment can be made at motorized platforms and/or the attachment points can be moveable by operation of connected motors. Responsive to operation of the motors the threaded rings can be positioned along the length of the surgical device (e.g., along an x-axis). Surgical tools connected to the interior of the rings (3102, 3104, and 3106) can be positioned at any point along the circumference of the ring. In some examples the tools are connected to a motor, a motorized platform, etc., that is configured to travel the circumference of respective rings.

According to another embodiment, the device 3100 can be configured to perform a total knee joint replacement. The surgical actuators are programmed to perform the total knee replacement via sequences of steps with frequent rescanning of the operating surface, re-calibration of the instructions responsive to any changes in operating surface topology, and ultimately instructions for closing any incisions made.

FIG. 32 is a block diagram of a special purpose computer system that can be specially programmed and/or configured to control surgical actuators. The computer system can be configured to receive sensor information to map a topology of an operating surface or surgical field. Using the topology the system can implement surgical instructions, select surgical tools, and execute the surgical instructions responsive to depth determined from the instructions and/or the topology. In some embodiments, the system links operation of the surgical actuators and/or print heads to reference guides which provide a zero depth reference or a zero z-axis position. In some embodiments, the system is configured to received image data and translate the image data into print instructions for the device responsive to mapping the image data to the topology of the operating surface. In further embodiments, the system can be configured to determine how large a print area is required, including determining and providing feedback to a user on how many repositionings of the device will be required to complete printing of an image.

Referring to FIG. 32, there is illustrated a block diagram of a distributed computer system 3200, in which various aspects and functions are practiced. As shown, the distributed computer system 3200 includes one or more computer systems that exchange information. More specifically, the distributed computer system 3200 includes computer systems 3202, 3204 and 3206. As shown, the computer systems 3202, 3204 and 3206 are interconnected by, and may exchange data through, a communication network 3208.

In some embodiments, the network 3208 may include any communication network through which computer systems may exchange data. To exchange data using the network 3208, the computer systems 3202, 3204 and 3206 and the network 3208 may use various methods, protocols and standards, including, among others, Fibre Channel, Token Ring, Ethernet, Wireless Ethernet, Bluetooth, IP, IPV6, TCP/IP, UDP, DTN, HTTP, FTP, SNMP, SMS, MMS, SS7, JSON, SOAP, CORBA, REST and Web Services. To ensure data transfer is secure, the computer systems 3202, 3204 and 3206 may transmit data via the network 3208 using a variety of security measures including, for example, TLS, SSL or VPN. While the distributed computer system 3200 illustrates three networked computer systems, the distributed computer system 3200 is not so limited and may include any number of computer systems and computing devices, networked using any medium and communication protocol.

As illustrated in FIG. 32, the computer system 3202 includes a processor 3210, a memory 3212, a bus 3214, an interface 3216 and data storage 3218. To implement at least some of the aspects, functions and processes disclosed herein, the processor 3210 performs a series of instructions that result in manipulated data. The processor 3210 may be any type of processor, multiprocessor or controller. Some exemplary processors include commercially available processors such as an Intel Xeon, Itanium, Core, Celeron, or Pentium processor, an AMD Opteron processor, a Sun UltraSPARC or IBM Power5+ processor and an IBM mainframe chip. The processor 3210 is connected to other system components, including one or more memory devices 3212, by the bus 3214.

The memory 3212 stores programs and data during operation of the computer system 3202. Thus, the memory 3212 may be a relatively high performance, volatile, random access memory such as a dynamic random access memory (DRAM) or static memory (SRAM). However, the memory 3212 may include any device for storing data, such as a disk drive or other non-volatile storage device. Various examples may organize the memory 3212 into particularized and, in some cases, unique structures to perform the functions disclosed herein. These data structures may be sized and organized to store values for particular data and types of data.

Components of the computer system 3202 are coupled by an interconnection element such as the bus 3214. The bus 3214 may include one or more physical busses, for example, busses between components that are integrated within a same machine, but may include any communication coupling between system elements including specialized or standard computing bus technologies such as IDE, SCSI, PCI and InfiniBand. The bus 3214 enables communications, such as data and instructions, to be exchanged between system components of the computer system 3202.

The computer system 3202 also includes one or more interface devices 3216 such as input devices, output devices and combination input/output devices. Interface devices may receive input or provide output. More particularly, output devices may render information for external presentation. Input devices may accept information from external sources. Examples of interface devices include keyboards, mouse devices, trackballs, microphones, touch screens, printing devices, display screens, speakers, network interface cards, etc. Interface devices allow the computer system 3202 to exchange information and to communicate with external entities, such as users and other systems.

The data storage 3218 includes a computer readable and writeable nonvolatile, or non-transitory, data storage medium in which instructions are stored that define a program or other object that is executed by the processor 3210. The data storage 3218 also may include information that is recorded, on or in, the medium, and that is processed by the processor 3210 during execution of the program. More specifically, the information may be stored in one or more data structures specifically configured to conserve storage space or increase data exchange performance.

The instructions stored in the data storage may be persistently stored as encoded signals, and the instructions may cause the processor 3210 to perform any of the functions described herein. The medium may be, for example, optical disk, magnetic disk or flash memory, among other options. In operation, the processor 3210 or some other controller causes data to be read from the nonvolatile recording medium into another memory, such as the memory 3212, that allows for faster access to the information by the processor 3210 than does the storage medium included in the data storage 3218. The memory may be located in the data storage 3218 or in the memory 3212, however, the processor 3210 manipulates the data within the memory, and then copies the data to the storage medium associated with the data storage 3218 after processing is completed. A variety of components may manage data movement between the storage medium and other memory elements and examples are not limited to particular data management components. Further, examples are not limited to a particular memory system or data storage system.

Although the computer system 3202 is shown by way of example as one type of computer system upon which various aspects and functions may be practiced, aspects and functions are not limited to being implemented on the computer system 3202 as shown in FIG. 32. Various aspects and functions may be practiced on one or more computers having a different architectures or components than that shown in FIG. 32. For instance, the computer system 3202 may include specially programmed, special-purpose hardware, such as an application-specific integrated circuit (ASIC) tailored to perform a particular operation disclosed herein. While another example may perform the same function using a grid of several general-purpose computing devices running MAC OS System X with Motorola PowerPC processors and several specialized computing devices running proprietary hardware and operating systems.

The computer system 3202 may be a computer system including an operating system that manages at least a portion of the hardware elements included in the computer system 3202. In some examples, a processor or controller, such as the processor 3210, executes an operating system. Examples of a particular operating system that may be executed include a Windows-based operating system, such as, Windows NT, Windows 2000 (Windows ME), Windows XP, Windows Vista or Windows 7 or 8 operating systems, available from the Microsoft Corporation, a MAC OS System X operating system available from Apple Computer, one of many Linux-based operating system distributions, for example, the Enterprise Linux operating system available from Red Hat Inc., a Solaris operating system available from Sun Microsystems, or a UNIX operating systems available from various sources. Many other operating systems may be used, and examples are not limited to any particular operating system.

The processor 3210 and operating system together define a computer platform for which application programs in high-level programming languages are written. These component applications may be executable, intermediate, bytecode or interpreted code which communicates over a communication network, for example, the Internet, using a communication protocol, for example, TCP/IP. Similarly, aspects may be implemented using an object-oriented programming language, such as .Net, SmallTalk, Java, C++, Ada, C# (C-Sharp), Objective C, or Javascript. Other object-oriented programming languages may also be used. Alternatively, functional, scripting, or logical programming languages may be used.

Additionally, various aspects and functions may be implemented in a non-programmed environment, for example, documents created in HTML, XML or other format that, when viewed in a window of a browser program, can render aspects of a graphical-user interface or perform other functions. For example, an administration component can render an interface in a browser to enable definition of contamination risks.

Further, various examples may be implemented as programmed or non-programmed elements, or any combination thereof. For example, a web page may be implemented using HTML while a data object called from within the web page may be written in C++. Thus, the examples are not limited to a specific programming language and any suitable programming language could be used. Accordingly, the functional components disclosed herein may include a wide variety of elements, e.g. specialized hardware, executable code, data structures or objects, which are configured to perform the functions described herein.

In some examples, the components disclosed herein may read parameters that affect the functions performed by the components. These parameters may be physically stored in any form of suitable memory including volatile memory (such as RAM) or nonvolatile memory (such as a magnetic hard drive). In addition, the parameters may be logically stored in a propriety data structure (such as a database or file defined by a user mode application) or in a commonly shared data structure (such as an application registry that is defined by an operating system). In addition, some examples provide for both system and user interfaces that allow external entities to modify the parameters and thereby configure the behavior of the components.

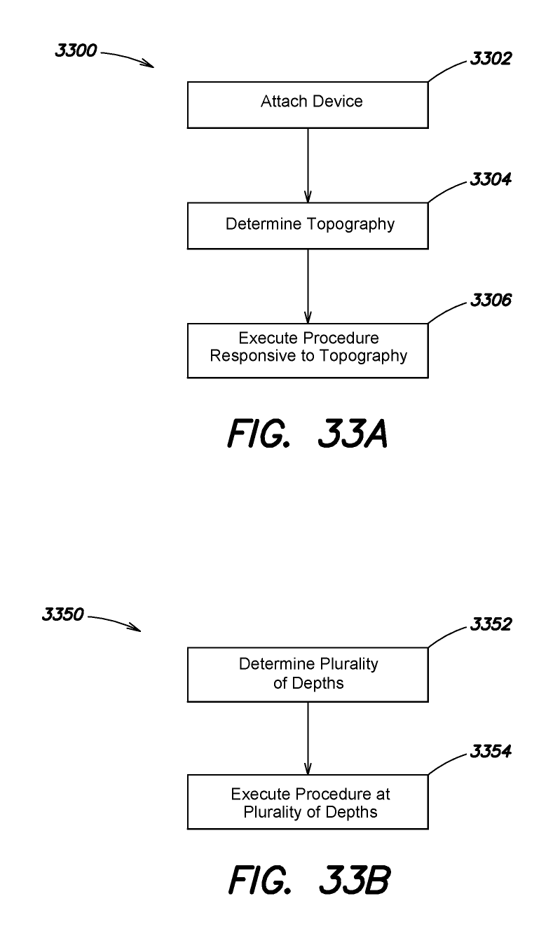

In FIG. 33A an example process flow for executing the procedure on a target body surface is illustrated. Process 3300 begins with attaching a wearable device above the target body surface at 3302. At 3304 the device determines the topography of the target surface. At 3306 the device automatically executes a procedure responsive to the determined topography. In one example, the device calibrated execution instructions for performing the procedure based on the determined topography.

In FIG. 33B an example process flow for executing the procedure on a target body surface is illustrated. Process 3350 begins at 3352 with determining a plurality of depths at which a procedure is to be executed on a target body surface. In some embodiments process 3350 can be executed as part of other processes (e.g., 3300). At 3354, once the plurality of depths has been determined, an automated procedure is executed at the plurality of depths.

It is to be appreciated that embodiments of the methods and apparatuses discussed herein are not limited in application to the details of construction and the arrangement of components set forth in the following description or illustrated in the accompanying drawings. The methods and apparatuses are capable of implementation in other embodiments and of being practiced or of being carried out in various ways. Examples of specific implementations are provided herein for illustrative purposes only and are not intended to be limiting. In particular, acts, elements and features discussed in connection with any one or more embodiments are not intended to be excluded from a similar role in any other embodiments.

Also, the phraseology and terminology used herein is for the purpose of description and should not be regarded as limiting. Any references to embodiments or elements or acts of the systems and methods herein referred to in the singular may also embrace embodiments including a plurality of these elements, and any references in plural to any embodiment or element or act herein may also embrace embodiments including only a single element. References in the singular or plural form are not intended to limit the presently disclosed systems or methods, their components, acts, or elements. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms.

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A wearable surgical device comprising:
an actuator coupled to at least one tool;
a plurality of motors for positioning the actuator in at least an x and y co-ordinate;
a reference guide configured to establish a distance to a target surface;
a driver operatively connected to the actuator for positioning the at least one tool in a z dimension, and
programming instructions configured to:
position the actuator based on programmatic activation of the plurality of motors,
position the tool based on programmatic activation of the driver, and
execute a procedure on a target surface based on programmatic action of one or more or the plurality of motors, the driver, and the tool, wherein the programmatic action is determined responsive to the position defined by the reference guide.

2. The device of claim 1, wherein the tool comprises a print head and the device further comprises an ink reservoir.

3. The device of claim 2, wherein the device executes programmatic instructions to print a first portion of an image on the target surface comprising a person's skin.

4. The device of claim 3, wherein the device further comprises a plurality of laser scanners.

5. The device of claim 4, wherein the device is configured to identify that the device has been attached to a new target surface and identify the first portion of the image responsive to signals received from the laser scanners.

6. The device of claim 5, wherein the device computes a second section of the image to print responsive to determining the new position relative to a former position or relative to the first portion of the image.

7. The device of claim 1, wherein the tool comprises a high frequency oscillating needle connected to an ink reservoir.

8. The device of claim 7, wherein the tool comprising a needle connected to an ink reservoir.

9. The device of claim 8, wherein the device is configured to generate an image at a subcutaneous position.

10. The device of claim 9, wherein the device is further configured to generate an image on a plurality of subcutaneous depths not visible to a human eye.

11. The device of claim 10, wherein the plurality of subcutaneous depth include at least a first image portion generated at a first layer depth and a second image portion generated at a second layer depth.

12. A programmable surgical device comprising:
a surgical actuator coupled to at least one tool;
a plurality of motors for positioning the surgical actuator in at least an x and y co-ordinate;
an attachment member for fixing the surgical device in position over a bodily surface;
a driver operatively connected to the surgical actuator for positioning the at least one tool in a z dimension, and
programming instructions configured to:
position the surgical actuator based on programmatic activation of the plurality of motors,
position the tool based on programmatic activation of the driver, and
execute a procedure at a depth defined by the z dimension based on programmatic action of one or more or the plurality of motors, the driver, and the surgical tool.

13. The device of claim 12, further comprising a reference guide configured to establish a depth reference on a target surface.

14. The device of claim 13, wherein the reference guide comprises a skid plate.

15. The device of claim 13, wherein the reference guide comprises a guide wheel.

16. The device of claim 15, wherein the guide wheel is deployable from a surgical actuator and is responsive to contact with the target surface.

17. The device of claim 12, further comprising a base portion for contacting a body surface or tissue surface.

18. The device of claim 17, wherein the attachment member is coupled to the base portion.

19. The device of claim 18, wherein attachment component comprises at least one strap extensible about a body part.

20. The device of claim 17, where the base portion further comprises an opening, and wherein the surgical tool access the body surface or the tissue surface through the opening to execute the surgical procedure.

21. The device of claim 12, further comprising a second surgical actuator coupled to at least a second tool.

22. The device of claim 21, further comprising:
a respective plurality of motors for positioning the second surgical actuator in at least an x and y plane; and
a second driver operatively connected to the second surgical actuator for positioning the at least the second surgical tool in a z plane.

23. The device of claim 12, further comprising a plurality of tools housed in a storage portion of the device.

24. The device of claim 23, wherein the tools comprising at least one, of a print head, a high frequency oscillating needle, scalpel, suture, needle and thread, or a stapler.

25. The device according to claim 12, wherein the programming instructions are further configured to position the surgical actuator to release the surgical tool in the storage portion of the device.

26. The device according to claim 25, wherein the programming instructions are further configured to couple the surgical actuator to a different surgical tool.

27. The device according to claim 26, wherein the programming instructions are further configured to move a scalpel through a volume of tissue to be removed.

28. A computer implemented method, the method comprising:
attaching a wearable device above a target body surface;
determining by the wearable device a topography for the target body surface;
deploying an automated tool responsive to the determined topography for the body surface; and
executing a procedure with the automated tool following contours of the determined topography.

29. The method of claim 28, wherein the automated tool includes a needle connected to an ink reservoir.

30. The method of claimer 29, wherein the act of executing the procedure includes determining a plurality of depths to generate at least a first image component at a first depth under the target body surface and at least a second image component at a second depth under the target body surface.

31. The method of claim 30, further comprising an act of determining the first and second depths such that at least one of the first and second image component is not visible by human sight.

32. The method of claim 29, wherein the automated tool include a scalpel and wherein the act of executing the procedure includes determining a depth of tissue to excise from the body surface.

33. The method of claim 32, further comprising an act of changing the automated tool for a suturing or cautery tool, and wherein the act of executing the procedure includes an act of closing any incision.
